(19) United States
(12) Patent Application Publication
(10) Pub. No.: US 2007/0066672 A1
(43) Pub. Date: Mar. 22, 2007

(54) Benzimidazole derivatives and use thereof as peptide deformylase inhibitors

(75) Inventors: Anders Hjelmencrantz, Malmo (SE); Patrizia Cali, Copenhagen S (DK); Thomas Groth, Vaerlose (DK); Christian E. Jensen, Copenhagen V (DK); Lars Naerum, Hellerup (DK)

Correspondence Address: EDWARDS & ANGELL, LLP, P.O. BOX 55874, BOSTON, MA, 02205, US

(73) Assignee: Arpida A/S, Frederiksberg C (DK)

(21) Appl. No.: 10/572,415

(22) PCT Filed: Oct. 8, 2004
(86) PCT No.: PCT/DK04/00679

§ 371 Date: Mar. 17, 2006

Related U.S. Application Data
(60) Provisional application No. 60/513,891, filed on Oct. 23, 2003.

(30) Foreign Application Priority Data
Oct. 22, 2003 (DK) PA 2003 01553

(51) Int. Cl. A61K 31/4184; C07D 403/02; C07D 235/14

(52) U.S. Cl. 514/394; 548/305.4; 548/309.7

(57) ABSTRACT

Benzimidazole compounds of the general formula (I) and pharmaceutically acceptable salts or esters thereof are peptide deformylase inhibitors useful in the treatment or prevention of infections and other diseases in which peptide deformylases are involved, especially in the treatment of bacterial and parasitic infections, for example infections fully or partly caused by microorganisms belonging to Staphylococcus, Enterococcus, Streptococcus, Haemophilus, Moraxella, Escherichia, Mycobacterium, Mycoplasma, Pseudomonas, Chlamydia, Rickettsia, Klebsiella, Shigella, Salmonella, Bordetella, Clostridium, helicobacter, Campylobacter, Legionella, or Neisseria.

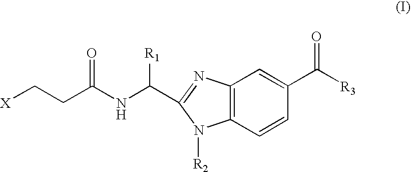

The present invention relates to novel enzyme inhibitors, more specifically to inhibitors of polypeptide deformylase useful in the treatment/prevention of infections and other diseases in which polypeptide deformylases are involved, especially in the treatment of bacterial and parasitic infections. More specifically the invention relates to benzimidazoles capable of inhibiting bacterial peptide deformylase, also known as PDF, an enzyme that catalyzes the deformylation of formyl-L-methionyl peptides.

### BACKGROUND OF THE INVENTION

Peptide deformylase (EC 3.4.1.88), also known as PDF, is an enzyme that catalyzes the deformylation of formyl-L-methionyl peptides. PDF removes the formyl group from the N-terminal Met of newly synthesized proteins, i.e. catalyzes the conversion of formyl-L-methionyl peptide to methionyl peptide (Adams and Capecchi, 1966; Adams, 1968). PDF is essential to bacteria, and bacterial peptide deformylase (PDF) is now widely recognised as an attractive target for antibacterial chemotherapy (Giglione et al., 2000; Giglione and Meinnel, 2001; Pei 2001; Yuan et al., 2001; Clements et al., 2002). Deformylation is a crucial step in bacterial protein biosynthesis and PDF is an essential ingredient in bacterial growth, with the gene encoding PDF present in all sequenced pathogenic bacterial genomes.

WO 02/41886A1 discloses hydroxamic acid or N-formyl hydroxylamine derivatives as inhibitors of bacterial polypeptide deformylase.

Novel antibacterial compounds are urgently needed due to the growing resistance exhibited by both Gram-negative and Gram-positive bacteria and other microorganisms. Traditional antibiotics have targeted pathways unique to bacterial replication and maintenance. However, new pathways are not being targeted in a manner that outpaces the growth of bacterial resistance. Thus, novel compounds and strategies are greatly needed that can be used to eradicate resistant bacteria.

### SUMMARY OF THE INVENTION

The present invention relates to compounds of the general formula (I)

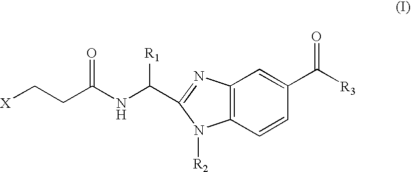

or a pharmaceutically acceptable salt or ester thereof,

wherein R₁, R₂, R₃, and X are as defined in the detailed part of this description.

It is contemplated that the compounds of the invention are useful for the treatment of infections caused by bacteria or parasites. It is especially contemplated that the compounds of the present invention are useful for the treatment of infections fully or partly caused by Gram-positive or Gram-negative bacteria such as Escherichia coli and Staphylococcus aureus or by a parasite such as Plasmodium falciparum.

It is an object of the invention to provide novel compounds having pharmacological activity as inhibitors of PDF.

Further objects will become apparent from the following description.

### DETAILED DESCRIPTION OF THE INVENTION

Definitions

The terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

The term “peptide deformylase” or “PDF” as used herein is intended to mean peptide deformylase (EC 3.4.1.88) also known as PDF, which catalyzes the conversion of the N-terminal formyl-L-methionyl peptide to methionyl peptide in newly synthesized proteins.

The term “treatment” is defined as the management and care of a patient for the purpose of combating the disease, condition, or disorder and includes the administration of a compound of the present invention to prevent the onset of the symptoms or the complications, or alleviating the symptoms or the complications, or eliminating the disease, condition, or disorder.

As used herein, alone or in combination, the term “C₁₋₆ alkyl” denotes a straight or branched, saturated hydrocarbon chain having from one to six carbon atoms. C₁₋₆ alkyl groups include, but are not limited to, methyl, ethyl, n-propyl, iso-propyl, n-butyl, iso-butyl, sec-butyl, tert-butyl, n-pentyl, iso-pentyl, 2-methylbutyl, 3-methylbutyl, n-hexyl, iso-hexyl, 4-methylpentyl, neopentyl, 2,2-dimethylpropyl and the like.

As used herein, alone or in combination, the term “C₂₋₆ alkenyl” denotes a straight or branched, unsaturated hydrocarbon chain having from two to six carbon atoms and at least one double bond. C₂₋₆ alkenyl groups include, but are not limited to, vinyl, 1-propenyl, allyl, iso-propenyl, n-butenyl, n-pentenyl, n-hexenyl and the like.

The term “C₁₋₆ alkoxy” in the present context designates a group —O—C₁₋₆ alkyl used alone or in combination, wherein C₁₋₆ alkyl is as defined above. Examples of linear alkoxy groups are methoxy, ethoxy, propoxy, butoxy, pentoxy and hexoxy. Examples of branched alkoxy are iso-propoxy, sec-butoxy, tert-butoxy, iso-pentoxy and iso-hexoxy.

Examples of cyclic alkoxy are cyclopropyloxy, cyclobutyloxy, cyclopentyloxy and cyclohexyloxy.

The term “C₃₋₁₀ cycloalkyl” as used herein denotes a radical of one or more saturated mono-, bi-, tri- or spirocyclic hydrocarbon having from three to ten carbon atoms. Examples include, but are not limited to, cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “C₃₋₁₀ cycloalkane” as used herein refers to a saturated cyclic hydrocarbon having from three to ten carbon atoms. Examples include, but are not limited to, cyclopropane, cyclobutane, cyclopentane, cyclohexane, adamantane and the like.

The term “C₃₋₇ heterocycloalkyl” as used herein denotes a radical of a totally saturated heterocycle like a cyclic hydrocarbon containing one or more heteroatoms selected from nitrogen, oxygen and sulphur independently in the cycle. Examples of heterocycles include, but are not limited to, pyrrolidine(1-pyrrolidine, 2-pyrrolidine, 3-pyrrolidine, 4-pyrrolidine, 5-pyrrolidine), pyrazolidine-(1-pyrazolidine, 2-pyrazolidine, 3-pyrazolidine, 4-pyrazolidine, 5-pyrazolidine), imidazolidine(1-imidazolidine, 2-imidazolidine, 3-imidazolidine, 4-imidazolidine, 5-imidazolidine), thiazolidine(2-thiazolidine, 3-thiazolidine, 4-thiazolidine, 5-thiazolidine), piperidine(1-piperidine, 2-piperidine, 3-piperidine, 4-piperidine, 5-piperidine, 6-piperidine), piperazine(1-piperazine, 2-piperazine, 3-piperazine, 4-piperazine, 5-piperazine, 6-piperazine), morpholine(2-morpholine, 3-morpholine, 4-morpholine, 5-morpholine, 6-morpholine), thiomorpholine(2-thiomorpholine, 3-thiomorpholine, 4-thiomorpholine, 5-thiomorpholine, 6-thiomorpholine), 1,2-oxathiolane(3-(1,2-oxathiolane), 4-(1,2-oxathiolane), 5-(1,2-oxathiolane)), 1,3-dioxolane(2-(1,3-dioxolane), 3-(1,3-dioxolane), 4-(1,3-dioxolane)), tetrahydropyrane(2-tetrahydropyrane, 3-tetrahydropyrane, 4-tetrahydropyrane, 5-tetrahydropyrane, 6-tetrahydropyrane), hexahydropyradizine, (1-(hexahydropyradizine), 2-(hexahydropyradizine), 3-(hexahydropyradizine), 4-(hexahydropyradizine), 5-(hexahydropyradizine), 6-(hexahydropyradizine)).

The term “C₁₋₆alkyl-C₃₋₁₀cycloalkyl” as used herein refers to a cycloalkyl group as defined above attached through an alkyl group as defined above having the indicated number of carbon atoms.

The term “C₁₋₆alkyl-C₃₋₇heterocycloalkyl” as used herein refers to a heterocycloalkyl group as defined above attached through an alkyl group as defined above having the indicated number of carbon atoms.

The term “aryl” as used herein is intended to include carbocyclic aromatic ring systems. Aryl is also intended to include the partially hydrogenated derivatives of the carbocyclic systems enumerated below.

The term “heteroaryl” as used herein includes heterocyclic unsaturated ring systems containing one or more heteroatoms selected among nitrogen, oxygen and sulphur, such as furyl, thienyl, pyrrolyl, and is also intended to include the partially hydrogenated derivatives of the heterocyclic systems enumerated below.

Examples of “aryl” and “heteroaryl” include, but are not limited to, phenyl, biphenyl, indenyl, naphthyl(1-naphthyl, 2-naphthyl), N-hydroxytetrazolyl, N-hydroxytriazolyl, N-hydroxyimidazolyl, anthracenyl(1-anthracenyl, 2-anthracenyl, 3-anthracenyl), phenanthrenyl, fluorenyl, pentalenyl, azulenyl, biphenylenyl, thiophenyl(1-thienyl, 2-thienyl), furyl(1-furyl, 2-furyl), furanyl, thiophenyl, isoxazolyl, isothiazolyl, 1,2,3-triazolyl, 1,2,4-triazolyl, pyranyl, pyridazinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl(thianaphthenyl), indolyl, oxadiazolyl, isoxazolyl, quinazolinyl, fluorenyl, xanthenyl, isoindanyl, benzhydryl, acridinyl, benzisoxazolyl, purinyl, quinazolinyl, quinolizinyl, quinolinyl, isoquinolinyl, quinoxalinyl, naphthyridinyl, phteridinyl, azepinyl, diazepinyl, pyrrolyl(2-pyrrolyl), pyrazolyl(3-pyrazolyl), imidazolyl(1-imidazolyl, 2-imidazolyl, 4-imidazolyl, 5-imidazolyl), triazolyl(1,2,3-triazol-1-yl, 1,2,3-triazol-2-yl, 1,2,3-triazol-4-yl, 1,2,4-triazol-3-yl), oxazolyl(2-oxazolyl, 4-oxazolyl, 5-oxazolyl), thiazolyl(2-thiazolyl, 4-thiazolyl, 5-thiazolyl), pyridyl(2-pyridyl, 3-pyridyl, 4-pyridyl), pyrimidinyl(2-pyrimidinyl, 4-pyrimidinyl, 5-pyrimidinyl, 6-pyrimidinyl), pyrazinyl, pyridazinyl(3-pyridazinyl, 4-pyridazinyl, 5-pyridazinyl), isoquinolyl(1-isoquinolyl, 3-isoquinolyl, 4-isoquinolyl, 5-isoquinolyl, 6-isoquinolyl, 7-isoquinolyl, 8-isoquinolyl), quinolyl(2-quinolyl, 3-quinolyl, 4-quinolyl, 5-quinolyl, 6-quinolyl, 7-quinolyl, 8-quinolyl), benzo[b]furanyl(2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl(2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl)), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl)), indolyl(1-indolyl, 2-indolyl, 3-indolyl, 4-indolyl, 5-indolyl, 6-indolyl, 7-indolyl), indazolyl(1-indazolyl, 2-indazolyl, 3-indazolyl, 4-indazolyl, 5-indazolyl, 6-indazolyl, 7-indazolyl), benzimidazolyl, (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl(1-benzoxazolyl, 2-benzoxazolyl), benzothiazolyl(1-benzothiazolyl, 2-benzothiazolyl, 4-benzothiazolyl, 5-benzothiazolyl, 6-benzothiazolyl, 7-benzothiazolyl), carbazolyl(1-carbazolyl, 2-carbazolyl, 3-carbazolyl, 4-carbazolyl). Non-limiting examples of partially hydrogenated derivatives are 1,2,3,4-tetrahydronaphthyl, 1,4-dihydronaphthyl, pyrrolinyl, pyrazolinyl, indolinyl, oxazolidinyl, oxazolinyl, oxazepinyl and the like.

The term “C₁₋₆ alkylaryl” as used herein refers to an aryl group as defined above attached through a C₁₋₆alkyl group as defined above having one, two, three, four, five or six carbon atoms; it is to be understood that the term includes unsubstituted or substituted C₁₋₆ alkylaryl.

The term “C₁₋₆ alkylheteroaryl” as used herein refers to a heteroaryl group as defined above attached through a C₁₋₆ alkyl group as defined above having one, two, three, four, five or six carbon atoms; it is to be understood that the term includes unsubstituted or substituted C₁₋₆ alkylheteroaryl.

The term “thioC₁₋₆-alkyl” in the present context designates a group —S—C₁₋₆-alkyl, wherein C₁₋₆-alkyl is as defined above. Representative examples include, but are not limited to, methylthio, ethylthio, n-propylthio, isopropylthio, butylthio, isobutylthio, sec-butylthio, tert-butylthio, n-pentylthio, isopentylthio, neopentylthio, tert-pentylthio, n-hexylthio, isohexylthio and the like.

The term “C₁₋₆ alkylthio-C₁₋₆ alkyl” in the present context designates a group —C₁₋₆alkyl-S—C₁₋₆alkyl, wherein C₁₋₆-alkyl is as defined above. Representative examples include, but are not limited to, methylthio methyl, ethylthio methyl (i;e. —CH₂—S—C₂H₅), n-propylthio methyl, isopropylthio methyl, butylthio methyl, isobutylthio methyl, sec-butylthio methyl, tert-butylthio methyl, n-pentylthio methyl, isopentylthio methyl, neopentylthio methyl, tert-pentylthio methyl, methylthio ethyl, methylthio propyl, methylthio isopropyl, methylthio butyl, methylthio isobutyl, methylthio pentyl, methylthio isopentyl, methylthio hexyl, methylthio isohexyl and the like.

The term “C₁₋₆alkylmercapto” in the present context designates a group —C₁₋₆-alkyl-SH, wherein C₁₋₆-alkyl is as defined above. Representative examples include, but are not limited to mercapto methyl (i.e. —CH₂—SH), mercapto ethyl, mercapto n-propyl, mercapto isopropyl, mercapto butyl, mercapto isobutyl, mercapto sec-butyl, mercapto tert-butyl, mercapto n-pentyl, mercapto isopentyl, mercapt neopentylo, mercapto tert-pentyl, mercapto n-hexyl, mercapto isohexyl and the like.

The term “C₁₋₆-alkylhydroxy” in the present context designates a group —C₁₋₆-alkyl-OH wherein C₁₋₆-alkyl is as defined above. Representative examples include, but are not limited to, methylhydroxy (i.e. —CH₂—OH), ethylhydroxy, n-propylhydroxy, isopropylhydroxy, butylhydroxy, isobutylhydroxy, sec-butylhydroxy, tert-butylhydroxy, n-pentylhydroxy, isopentylhydroxy, neopentylhydroxy, tert-pentylhydroxy, n-hexylhydroxy, isohexylhydroxy and the like.

The term “C₁₋₆ alkylcarboxy” in the present context designates a group —C₁₋₆-alkyl-COOH wherein C₁₋₆-alkyl is as defined above. Representative examples include, but are not limited to, carboxymethyl (i.e. —CH₂—COOH), carboxyethyl, carboxypropyl, carboxybutyl, carboxypentyl, carboxyhexyl and the like

The term “C₁₋₆alkylamide” in the present context designates a group —C₁₋₆alkyl-CONH₂, wherein C₁₋₆alkyl is as defined above. Representative examples include, but are not limited to, carbamoylmethyl (i.e. —CH₂—CONH₂), carbamoylethyl, carbamoylpropyl, carbamoylbutyl, carbamoylpentyl, carbamoylhexyl and the like.

The term “C₁₋₆-alkylamino” in the present context designates a group —C₁₋₆-alkyl-NH₂ wherein C₁₋₆-alkyl is as defined above. Representative examples include, but are not limited to, methylamino (i.e. —CH₂—NH₂), ethylamino, n-propylamino, isopropylamino, butylamino, isobutylamino, sec-butylamino, tert-butylamino, n-pentylamino, isopentylamino, neopentylamino, tert-pentylamino, n-hexylamino, isohexylamino and the like.

The term “alkylamino-C₁₋₆-alkyl” in the present context designates a group —C₁₋₆-alkyl-NH—C₁₋₆-alkyl wherein C₁₋₆-alkyl is as defined above. Representative examples include, but are not limited to, methylamino methyl, ethylamino methyl (i.e. —CH₂—NH—C₂H₅), n-propylamino methyl, isopropylamino methyl, butylamino methyl, isobutylamino methyl, sec-butylamino methyl, tert-butylamino methyl, n-pentylamino methyl, isopentylamino methyl, neopentylamino methyl, tert-pentylamino methyl, n-hexylamino methyl, isohexylamino methyl, methylamino ethyl, methylamino propyl, methylamino isopropyl, methylamino butyl, methylamino isobutyl, methylamino pentyl, methylamino isopentyl, methylamino hexyl, methylamino isohexyl and the like.

The term “dialkylamino-C₁₋₆alkyl” in the present context designates a group (C₁₋₆-alkyl)₂-N—C₁₋₆-alkyl wherein C₁₋₆-alkyl is as defined above. Representative examples include, but are not limited to, dimethylamino methyl, diethylamino methyl (i.e. —CH₂—N—(C₂H₅)₂), dipropylamino methyl, di-isopropylamino methyl, dibutylamino methyl, di-isobutylamino methyl, di-sec-butylamino methyl, di-tert-butylamino methyl, dipentylamino methyl, di-isopentylamino methyl, di-neopentylamino methyl, di-tert-pentylamino methyl, dihexylamino methyl, diisohexylamino methyl, dimethylamino ethyl, dimethylamino propyl, dimethylamino isopropyl, dimethylamino butyl, dimethylamino isobutyl, dimethylamino pentyl, dimethylamino isopentyl, dimethylamino hexyl, dimethylamino isohexyl and the like.

The term “C₁₋₆alkylamidine” in the present context designates a group —C₁₋₆-alkyl-C(═NH)NH₂, wherein C₁₋₆-alkyl is as defined above. Representative examples include, but are not limited to, methylamidine, ethylamidine, propylamidine, butylamidine, pentylamidine, hexylamidine and the like.

The term “C₁₋₆alkylguanidine” in the present context designates a group —C₁₋₆-alkyl-N—C(═NH)NH₂, wherein C₁₋₆alkyl-is as defined above. Representative examples include, but are not limited to, 1-methylguanidine, 1-ethylguanidine, 1-propylguanidine, 1-butylguanidine, 1-pentylguanidine, 1-hexylguanidine and the like.

“Halogen” designates an atom selected from the group consisting of F, Cl, Br and I.

The terms “unsubstituted” or “substituted” as used herein means that the groups in question are optionally unsubstituted or substituted with one, two or three substituents independently of each other selected from halogen, hydroxy, amino, mercapto, nitro, cyano, trifluoromethyl, trifluoromethylthio, trifluoromethoxy, C₁₋₆alkyl, C₁₋₆alkoxy, thioC₁₋₆-alkyl, C₁₋₆alkylamino, alkylamino-C₁₋₆alkyl and dialkylamino-C₁₋₆alkyl. When the groups in question are substituted with more than one substituent the substituents may be the same or different.

The terms “amino acid”, “amino acid residue”, “natural amino acid” and “natural amino acid residue” as used herein all refer to the D- or L-isomers of the more than 20 standard amino acid residues including alanine (Ala), arginine (Arg), asparagine (Asn), aspartic acid (Asp), cysteine (Cys), glutamine. (Gin), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val). An amino acid is a monomer containing an amino group and a carboxyl group that can be polymerized to form peptide and protein chains. Typically, peptide/protein-forming amino acids have the amino and carboxyl groups attached to the same carbon atom (the alpha carbon) and are designated alpha amino acids. The term “side chain of an alpha amino acid” denotes the substituent on the alpha carbon. Variable substituents generate different amino acids with different chemical properties. An amino acid residue is the portion of the amino acid that remains after incorporation into a polypeptide chain. The residue includes the alpha-carbon and the nitrogen/carbonyl moieties.

Certain of the above defined terms may occur more than once in the structural formula, and upon such occurrence each term shall be defined independently of the other.

As used herein, the phrase “a functional group which can be converted to hydrogen in vivo” is intended to include any group which upon administering the present compounds to the subjects in need thereof can be converted to hydrogen e.g. enzymatically or by the acidic environment in the stomach. Non-limiting examples of such groups are acyl, carbamoyl, monoalkylated carbamoyl, dialkylated carbamoyl, alkoxycarbonyl, alkoxyalkyl groups and the like such as C₁₋₆-alkylcarbonyl, aroyl, C₁₋₆-alkylcarbamoyl, di-C₁₋₆ alkyl-alkylcarbamoyl, C₁₋₆-alkoxycarbonyl and C₁₋₆-alkoxy-C₁₋₆-alkyl.

As used herein, the phrase “diseases and disorders related to peptide deformylase” is intended to include any disease or disorder in which an effect, preferably an inhibiting effect, on peptide deformylase is beneficial, especially on the bacterial peptide deformylase.

The term “IC₅₀” as used herein denotes the concentration required for 50% inhibition of PDF in a binding assay.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

The Compounds

The present invention relates to compounds of the general formula (I)

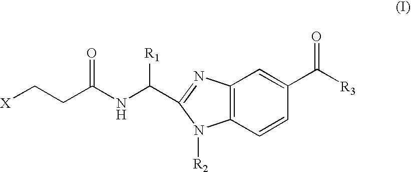

or a pharmaceutically acceptable salt or ester thereof,

wherein

X is —CONHOH, —COOH, —OH, or —SH;

R₁ is selected from the group consisting of C₁₋₆ alkyl, C₃₋₁₀ cycloalkyl, C₁₋₆ alkylmercapto, C₁₋₆ alkylthio-C₁₋₆ alkyl, C₁₋₆ alkylhydroxy, C₁₋₆ alkylcarboxy, C₁₋₆alkylamide, C₁₋₆ alkylamino, alkylamino-C₁₋₆alkyl, dialkylamino-C₁₋₆alkyl, C₁₋₆alkylamidine, C₁₋₆alkylguanidine, an unsubstituted or substituted aryl group, an unsubstituted or substituted heteroaryl group, an unsubstituted or substituted C₁₋₆ alkylaryl group, an unsubstituted or substituted C₁₋₆ alkylheteroaryl group and a side chains of a natural alpha amino acid;

with the proviso that R₁ cannot be hydrogen or tert-butyl;

R₂ is selected from the group consisting of C₁₋₆ alkyl, C₂₋₆ alkenyl, C₃₋₁₀ cycloalkyl, C₁₋₆ alkyl-C₃₋₁₀ cycloalkyl, C₃₋₇ heterocycloalkyl, C₁₋₆ alkoxy, C₁₋₆ alkylamino, C₁₋₆ alkylmercapto, C₁₋₆ alkylhydroxy, thioC₁₋₆ alkyl, alkylamino-C₁₋₆alkyl, dialkylamino-C₁₋₆alkyl, an unsubstituted or substituted aryl group, an unsubstituted or substituted heteroaryl group, an unsubstituted or substituted C₁₋₆ alkylaryl group and an unsubstituted or substituted C₁₋₆ alkylheteroaryl group;

R₃ is —NHCH(R₄)COR₅, —NR₆R₇, —NHR₇ or —OR₇;

R₄ is selected from the group consisting of hydrogen and a side chain of a natural alpha amino acid;

R₅ is amino, hydroxy, C₁₋₆ alkoxy or —NH—C₁₋₆alkyl;

R₆ and R₇ are identical or different and are independently of each other selected from the group consisting of C₃₋₇ heterocycloalkyl, an unsubstituted or substituted C₁₋₆ alkyl-C₃₋₇ heterocycloalkyl group, an unsubstituted or substituted aryl group, an unsubstituted or substituted heteroaryl group, an unsubstituted or substituted C₁₋₆ alkylaryl group and an unsubstituted or substituted C₁₋₆ alkylheteroaryl group;

wherein a substituted group is substituted with one, two or three substituents independently selected from halogen, hydroxy, amino, mercapto, nitro, cyano, trifluoromethyl, C₁₋₆ alkyl, C₁₋₆ alkoxy, thioC₁₋₆ alkyl, C₁₋₆ alkylhydroxy, C₁₋₆ alkylamino, alkylamino-C₁₋₆alkyl and dialkylamino-C₁₋₆alkyl.

In a preferred embodiment of the invention, X is —CONHOH or —COOH. However, in other useful compounds of the invention, X is —OH or —SH.

Preferably, R₁ is a side chain of a natural alpha amino acid such as alanine, arginine, asparagine, aspartic acid, cysteine, glutamine, glutamic acid, glycine, histidine, isoleucine, leucine, lysine, methionine, phenylalanine, serine, threonine, tryptophan, tyrosine and valine, or alternatively, R₁ is selected from the group consisting of C₁₋₆ alkyl, C₃₋₁₀ cycloalkyl, C₁₋₆ alkylmercapto, C₁₋₆ alkylthio-C₁₋₆ alkyl, C₁₋₆ alkylhydroxy, C₁₋₆ alkylcarboxy, C₁₋₆alkylamide, C₁₋₆ alkylamino, alkylamino-C₁₋₆alkyl, dialkylamino-C₁₋₆alkyl, C₁₋₆alkylamidine, C₁₋₆alkylguanidine, an unsubstituted or substituted aryl group, an unsubstituted or substituted heteroaryl group, an unsubstituted or substituted C₁₋₆ alkylaryl group and an unsubstituted or substituted C₁₋₆ alkylheteroaryl group. More preferably, R₁ is ethyl, isobutyl, 2-(methylsulfanyl)ethyl, 4-aminobutyl, benzyl, 4-hydroxybenzyl, 2-phenylethyl and naphth-1-yl-methyl.

R₂ is preferably selected from the group consisting of C₁₋₆ alkyl, C₃₋₁₀cycloalkyl, C₁₋₆ alkyl-C₃₋₁₀ cycloalkyl, C₁₋₆ alkylamino, C₁₋₆ alkylhydroxy, an unsubstituted or substituted C₁₋₆ alkylaryl group and an unsubstituted or substituted C₁₋₆ alkylheteroaryl group, wherein a substituted group is-substituted with one, two or three substituents independently selected from halogen, hydroxy, amino, mercapto, nitro, cyano, trifluoromethyl, C₁₋₆ alkyl, C₁₋₆ alkoxy and thioC₁₋₆ alkyl. More preferably, R₂ is selected from the group consisting of ethyl, propyl, butyl, cyclopropyl, cyclobutyl, cyclopentyl, cyclohexyl, cyclopropylmethyl, cyclobutylmethyl, cyclohexylmethyl, cyclohexylethyl, aminoethyl, aminopropyl, aminobutyl, hydroxymethyl, hydroxyethyl, hydroxypropyl, hydroxybutyl, an phenyl, fluorosubstituted phenyl, chlorosubstituted phenyl, benzyl, fluorosubstituted benzyl, chlorosubstituted benzyl, thiophenylethyl and furanylmethyl.

In particular R₂ may be selected from butyl, cyclopropyl, cyclohexylmethyl, 2-aminoethyl, 2-hydroxyethyl, benzyl, 2-chlorobenzyl, 4-chlorobenzyl, 2,6-difluorobenzyl, 2-thiophen-2-ylethyl or furan-2-ylmethyl.

In a preferred embodiment of the invention, R₃ is —NHCH(R₄)COR₅, in which R₄ is hydrogen or a side chain of a natural alpha amino acid, such as a side chain of alanine, arginine, asparagine, aspartic acid, cysteine, glutamine, glutamic acid, glycine, histidine, isoleucine, leucine, lysine, methionine, phenylalanine, serine, threonine, tryptophan, tyrosine or valine, preferably R₄ is hydrogen; and R₅ is C₁₋₆alkoxy, preferably methoxy, ethoxy, propoxy or butoxy.

In another preferred embodiment of the invention, R₃ is a group —NHR₇, a group —NR₆R₇ or a group —OR₇, in which R₆ or R₇ is C₃₋₇ heterocycloalkyl or an unsubstituted or substituted C₁₋₆ alkyl-C₃₋₇ heterocycloalkyl group. Alternatively, R₆ or R₇ is an unsubstituted or substituted aryl group, an unsubstituted or substituted heteroaryl group, an unsubstituted or substituted C₁₋₆ alkylaryl group or an unsubstituted or substituted C₁₋₆alkylheteroaryl group.

Preferred compounds of the invention are:

({1-cyclopropyl-2-[1-(3-mercapto-propionylamino)-propyl]-1H-benzoimidazole-5-carbonyl}-amino)-acetic acid methyl ester,

({1-(4-chloro-benzyl)-2-[1-(3-mercapto-propionylamino)-2-phenyl-ethyl]-1H-benzoimidazole-5-carbonyl}-amino)-acetic acid methyl ester,

N-{1-[1-benzyl-5-(methoxycarbonylmethyl-carbamoyl)-1H-benzoimidazol-2-yl]-3-methylsulfanyl-propyl}-succinamic acid,

N-{1-[1-butyl-5-(methoxycarbonylmethyl-carbamoyl)-1H-benzoimidazol-2-yl]-2-phenyl-ethyl}-succinamic acid,

N-{1-[1-furan-2-ylmethyl-5-(methoxycarbonylmethyl-carbamoyl)-1H-benzoimidazol-2-yl]-2-phenyl-ethyl}succinamic acid,

N-{1-[1-(4-chloro-benzyl)-5-(methoxycarbonylmethyl-carbamoyl)-1H-benzoimidazol-2-yl]-2-phenyl-ethyl}succinamic acid,

N-{1-[1-cyclopropyl-5-(methoxycarbonylmethyl-carbamoyl)-1H-benzoimidazol-2-yl]-3-phenyl-propyl}-succinamic acid,

N-{1-[1-cyclohexylmethyl-5-(methoxycarbonylmethyl-carbamoyl)-1H-benzoimidazol-2-yl]-3-phenyl-propyl}-succinamic acid,

N-{1-[1-(2-chloro-benzyl)-5-(methoxycarbonylmethyl-carbamoyl)-1H-benzoimidazol-2-yl]-3-phenyl-propyl}-succinamic acid,

N-{1-[1-cyclopropyl-5-(methoxycarbonylmethyl-carbamoyl)-1H-benzoimidazol-2-yl]-propyl}-succinamic acid,

N-{1-[1-furan-2-ylmethyl-5-(methoxycarbonylmethyl-carbamoyl)-1H-benzoimidazol-2-yl]-propyl}-succinamic acid,

N-{1-[1-benzyl-5-(methoxycarbonylmethyl-carbamoyl)-1H-benzoimidazol-2-yl]-propyl}-succinamic acid,

N-{1-[1-cyclopropyl-5-(methoxycarbonylmethyl-carbamoyl)-1H-benzoimidazol-2-yl]-3-methyl-butyl}-succinamic acid,

N-{1-[1-butyl-5-(methoxycarbonylmethyl-carbamoyl)-1H-benzoimidazol-2-yl]-3-methyl-butyl}-succinamic acid,

N-{1-[1-benzyl-5-(methoxycarbonylmethyl-carbamoyl)-1H-benzoimidazol-2-yl]-3-methyl-butyl}-succinamic acid,

N-{1-[1-cyclopropyl-5-(methoxycarbonylmethyl-carbamoyl)-1H-benzoimidazol-2-yl]-3-methylsulfanyl-propyl}-succinamic acid,

N-{1-[5-(methoxycarbonylmethyl-carbamoyl)-1-(2-thiophen-2-yl-ethyl)-1H-benzoimidazol-2-yl]-2-naphthalen-1-yl-ethyl}-succinamic acid,

N-{1-[1-butyl-5-(methoxycarbonylmethyl-carbamoyl)-1H-benzoimidazol-2-yl]-2-naphthalen-1-yl-ethyl}-succinamic acid,

({2-[5-amino-1-(3-mercapto-propionylamino)-pentyl]-1-cyclohexylmethyl-1H-benzoimidazole-5-carbonyl}-amino)-acetic acid methyl ester,

({1-cyclopropyl-2-[2-(4-hydroxy-phenyl)-1-(3-mercapto-propionylamino)-ethyl]-1H-benzoimidazole-5-carbonyl}-amino)-acetic acid methyl ester,

({1-cyclohexylmethyl-2-[2-(4-hydroxy-phenyl)-1-(3-mercapto-propionylamino)-ethyl]-1H-benzoimidazole-5-carbonyl}-amino)-acetic acid methyl ester,

({1-(2-hydroxy-ethyl)-2-[1-(3-mercapto-propionylamino)-2-phenyl-ethyl]-1H-benzoimidazole-5-carbonyl}-amino)-acetic acid methyl ester,

N-{5-amino-1-[1-cyclopropyl-5-(methoxycarbonylmethyl-carbamoyl)-1H-benzoimidazol-2-yl]-pentyl}-succinamic acid,

N-{5-amino-1-[1-cyclohexylmethyl-5-(methoxycarbonylmethyl-carbamoyl)-1H-benzoimidazol-2-yl]-pentyl}succinamic acid,

N-[1-[1-cyclopropyl-5-(methoxycarbonylmethyl-carbamoyl)-1H-benzoimidazol-2-yl-2-(4-hydroxyphenyl)-ethyl]-succinamic acid,

N-[1-[1-cyclohexylmethyl-5-(methoxycarbonylmethyl-carbamoyl)-1H-benzoimidazol-2-yl]-2-(4-hydroxyphenyl)-ethyl]-succinamic acid,

N-{1-[1-(2-hydroxy-ethyl)-5-(methoxycarbonylmethyl-carbamoyl)-1H-benzoimidazol-2-yl]-2-phenyl-ethyl}-succinamic acid,

N-{1-[1-(2-hydroxy-ethyl)-5-(methoxycarbonylmethyl-carbamoyl)-1H-benzoimidazol-2-yl]-3-methylbutyl}-succinamic acid,

({1-cyclopropyl-2-[1-(3-hydroxycarbamoyl-propionylamino)-propyl]-1H-benzoimidazole-5-carbonyl}-amino)-acetic acid methyl ester,

N-{1-[1-benzyl-5-(methoxycarbonylmethyl-carbamoyl)-1H-benzoimidazol-2-yl]-2-naphthalen-1-yl-ethyl}-succinamic acid,

({1-furan-2-ylmethyl-2-[1-(3-hydroxycarbamoyl-propionylamino)-propyl]-1H-benzoimidazole-5-carbonyl}-amino)-acetic acid methyl ester,

({1-benzyl-2-[1-(3-hydroxycarbamoyl-propionylamino)propyl]-1H-benzoimidazole-5-carbonyl}-amino)-acetic acid methyl ester,

({1-cyclopropyl-2-[1-(3-hydroxycarbamoyl-propionylamino)-3-methyl-butyl]-1H-benzoimidazole-5-carbonyl}-amino)-acetic acid methyl ester,

({1-butyl-2-[1-(3-hydroxycarbamoyl-propionylamino)-3-methyl-butyl]-1H-benzoimidazole-5-carbonyl}-amino)-acetic acid methyl ester,

({1-benzyl-2-[1-(3-hydroxycarbamoyl-propionylamino)-3-methyl-butyl]-1H-benzoimidazole-5-carbonyl}-amino)-acetic acid methyl ester,

({1-cyclopropyl-2-[1-(3-hydroxycarbamoyl-propionylamino)-3-methylsulfanyl-propyl]-1H-benzoimidazole-5-carbonyl}-amino)-acetic acid methyl ester,

({1-cyclohexylmethyl-2-[1-(3-hydroxycarbamoyl-propionylamino)-3-methylsulfanyl-propyl]-1H-benzoimidazole-5-carbonyl}-amino)-acetic acid methyl ester,

({1-benzyl-2-[1-(3-hydroxycarbamoyl-propionylamino)-3-methylsulfanyl-propyl]-1H-benzoimidazole-5-carbonyl}-amino)-acetic acid methyl ester,

({1-furan-2-ylmethyl-2-[1-(3-hydroxycarbamoyl-propionylamino)-2-phenyl-ethyl]-1H-benzoimidazole-5-carbonyl}-amino)-acetic acid methyl ester,

({1-(4-chloro-benzyl)-2-[1-(3-hydroxycarbamoyl-propionylamino)-2-phenyl-ethyl]-1H-benzoimidazole-5-carbonyl}-amino)-acetic acid methyl ester,

({1-cyclopropyl-2-[1-(3-hydroxycarbamoyl-propionylamino)-3-phenyl-propyl]-1H-benzoimidazole-5-carbonyl}-amino)-acetic acid methyl ester,

({1-cyclohexylmethyl-2-[1-(3-hydroxycarbamoyl-propionylamino)-3-phenyl-propyl]-1H-benzoimidazole-5-carbonyl}-amino)-acetic acid methyl ester,

({1-(2-chloro-benzyl)-2-[1-(3-hydroxycarbamoyl-propionylamino)-2-phenyl-ethyl]-1H-benzoimidazole-5-carbonyl}-amino)-acetic acid methyl ester,

{[2-[1-(3-hydroxycarbamoyl-propionylamino)-2-naphthalen-1-yl-ethyl]-1-(2-thiophen-2-yl-ethyl)-1H-benzoimidazole-5-carbonyl]-amino}-acetic acid methyl ester,

({1-butyl-2-[1-(3-hydroxycarbamoyl-propionylamino)-2-naphthalen-1-yl-ethyl]-1H-benzoimidazole-5-carbonyl}-amino)-acetic acid methyl ester,

({1-benzyl-2-[1-(3-hydroxycarbamoyl-propionylamino)-2-naphthalen-1-yl-ethyl]-1H-benzoimidazole-5-carbonyl}-amino)-acetic acid methyl ester,

({1-butyl-2-[1-(3-hydroxycarbamoyl-propionylamino)-2-phenyl-ethyl]-1H-benzoimidazole-5-carbonyl}-amino)-acetic acid methyl ester,

({2-[5-amino-1-(3-mercapto-propionylamino)-pentyl]-1-cyclopropyl-1H-benzoimidazole-5-carbonyl}-amino)-acetic acid methyl ester,

({2-[5-amino-1-(3-mercapto-propionylamino)-pentyl]-1-benzyl-1H-benzoimidazole-5-carbonyl}-amino)-acetic acid methyl ester,

({1-(2-amino-ethyl)-2-[1-(3-mercapto-propionylamino)-2-phenyl-ethyl]-1H-benzoimidazole-5-carbonyl}-amino)-acetic acid methyl ester,

N-{5-amino-1-[1-benzyl-5-(methoxycarbonylmethyl-carbamoyl)-1H-benzoimidazol-2-yl]-pentyl}-succinamic acid,

N-{1-[1-(2-amino-ethyl)-5-(methoxycarbonylmethyl-carbamoyl)-1H-benzoimidazol-2-yl]-2-phenyl-ethyl}-succinamic acid,

({2-[5-amino-1-(3-hydroxycarbamoyl-propionylamino)-pentyl]-1-cyclopropyl-1H-benzoimidazole-5-carbonyl}-amino)-acetic acid methyl ester,

({2-[5-amino-1-(3-hydroxycarbamoyl-propionylamino)-pentyl]-1-cyclohexylmethyl-1H-benzoimidazole-5-carbonyl}-amino)-acetic acid methyl ester,

({2-[5-amino-1-(3-hydroxycarbamoyl-propionylamino)-pentyl]-1-benzyl-1H-benzoimidazole-5-carbonyl}-amino)-acetic acid methyl ester,

({1-(2-amino-ethyl)-2-[1-(3-hydroxycarbamoyl-propionylamino)-2-phenyl-ethyl]-1H-benzoimidazole-5-carbonyl}-amino)-acetic acid methyl ester,

({1-cyclopropyl-2-[1-(3-hydroxycarbamoyl-propionylamino)-2-(4-hydroxy-phenyl)-ethyl]-1H-benzoimidazole-5-carbonyl}-amino)-acetic acid methyl ester,

({1-cyclohexylmethyl-2-[1-(3-hydroxycarbamoyl-propionylamino)-2-(4-hydroxy-phenyl)-ethyl]-1H-benzoimidazole-5-carbonyl}-amino)-acetic acid methyl ester,

{[2-[1-(3-hydroxycarbamoyl-propionylamino)-2-phenyl-ethyl]-1-(2-hydroxy-ethyl)-1H-benzoimidazole-5-carbonyl]-amino}-acetic acid methyl ester,

and stereoisomers thereof.

The compounds of the invention may exist as geometric isomers or optical isomers or stereoisomers as well as tautomers. Accordingly, the invention includes all geometric isomers and tautomers including mixtures and racemic mixtures of these and a pharmaceutically acceptable salt thereof, especially all R— and S-isomers. The compounds of the invention may also exist as solvent complexes as well as in different morphological forms.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates and solvent complexes, which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the -required compound of the Formula I. Prodrugs are any covalently bonded compounds, which release the active parent drug according to Formula l in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The present invention includes all complexes of the compounds of this invention.

The meaning of any substituent at any one occurrence in Formula I or any subformula thereof is independent of its meaning, or any other substituent's meaning, at any other occurrence, unless specified otherwise.

In a preferred embodiment of this invention, the compounds of Formula I exhibit in a bacterial PDF assay (see below) an IC₅₀ value of less than 500 μM, preferably less than 100 μM, more preferably less than 50 μM, even more preferably less than 1 μM, especially less than 500 nM, particularly 300 nM or less.

Synthetic Method of Preparation

The compounds of the present invention having the general Formula I may be prepared by the methods set forth in the scheme A and scheme B in ‘Materials and Method’ below.

Compounds wherein R₃ is —NHCH(R₄)COR₅ can be synthesized as depicted in scheme A. The aromatic core is coupled to the resin by standard procedures (step 1). Nucleophilic aromatic substitution by the proper amine affords the desired substituted aminonitrobenzoic acid bound to the resin (step 2). Reduction of the nitro functionality is achieved by using SnCl₂ in a suitable solvent like DMF (step 3). Amino acid coupling to the diaminobenzoic acid is achived by standard procedures (HATU, HOAt) to afford the desired product (step 4). Ring closure to produce the benzimidazole is obtained by heating the resin in neat acetic acid (step 5). The amino acid residue is deprotected by standard procedures, and coupling of the respective end functionality (CONHOH, COOH, OH or SH) is achieved by standard coupling procedures (TBTU, DIEA) (step 6). Finally the target molecule is cleaved off the resin by treatment with base (step 7).

Compounds wherein R₃ is —NHR₇, —NR₆R₇ or —OR₇ can be synthesized as depicted in scheme B. Nucleophilic aromatic substitution by the proper amine affords the desired substituted aminonitrobenzoic acid ester (step 1). Reduction of the nitro functionality is achieved by using NaBH₄/Cu(acac)₂ (step 2). Amino acid coupling to the diaminobenzoic acid ester is achived by standard procedures (HATU, HOAt) to afford the desired product (step 3). Ring closure to produce the benzimidazole is obtained by heating in neat acetic acid (step 4). The amino acid residue is deprotected by standard procedures, and coupling of the respective end functionality (CONHOH, COOH, OH or SH) is achieved by standard coupling procedures (TBTU, DIEA) (step 5). The corresponding primary amides are produced by aminolysis in NH₃/MeOH (step 6). The corresponding esters or amides are produced by basic hydrolysis of the methylester and subsequent coupling of amines using standard procedure (TBTU, NEM) or esterification by coupling of alcohols using standard procedure (MSNT, Methylimidazole) (step 7).

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions, which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

Pharmaceutical Compositions

In one aspect of this invention, there is provided a pharmaceutical composition comprising, as an active ingredient, a compound of the present invention together with a pharmaceutically acceptable carrier or diluent. This composition may be in unit dosage form and may comprise from about 1 μg to about 1000 mg such as, e.g., from about 10 μg to about 500 mg, from about 0.05 to about 100 mg or from about 0.1 to about 50 mg, of the compound of the invention or a pharmaceutically acceptable salt or ester thereof. The composition of the invention may be used for oral, nasal, transdermal, pulmonal or parenteral administration. It is contemplated that the pharmaceutical composition of the invention is useful for treatment of bacterial and/or parasitic infections.

The compounds of the invention may be administered alone or in combination with pharmaceutically acceptable carriers, diluents or excipients, in either single or multiple doses. Accordingly, the compounds of Formula I may be used in the manufacture of a medicament. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19.sup.th Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 50 mg/kg body weight per day, preferably from about 0.01 to about 30 mg/kg body weight per day, and more preferred from about 0.05 to about 20 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from about 1 μg to about 1000 mg such as, e.g., from about 10 μg to about 500 mg, 0.05 to about 500 mg, preferably from about 0.05 to about 100 mg, more preferably from about 0.1 to about 50 mg.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the Formula (I) contains a free base such salts are prepared in a conventional manner by treating a solution or suspension of a free base of the Formula (I) with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the novel compounds of the Formula (I) in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the novel compounds of the Formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tableted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet, which may be prepared by conventional tabletting techniques, may contain:

| Core: | | |
|---|---|---|
| Active compound (free compound or salt) | 5.0 | mg |
| Lactosum Ph. Eur. | 67.8 | mg |
| Cellulose, microcryst. (Avicel) | 31.4 | mg |
| Amberlite | 1.0 | mg |
| Magnesii stearas | q.s. | |
| Coating: | | |
| Hydroxypropyl methylcellulose approx. | 9 | mg |
| Acylated monoglyceride approx. | 0.9 | mg |

If desired, the pharmaceutical composition of the invention may comprise the compound of the Formula (I) in combination with further pharmacologically active substances such as those described in the foregoing.

Use of the Invention

The compounds of Formula I are useful as protease inhibitors, particularly as inhibitors of metallo proteases, more particularly as inhibitors of peptide deformylase, even more particularly as inhibitors of bacterial peptide deformylase. The present invention provides useful compositions and formulations of said compounds, including pharmaceutical compositions and formulations of said compounds.

The compounds of the present invention may be especially useful for the treatment or prevention of diseases caused by a variety of bacterial or prokaryotic organisms. Examples include Gram-positive and Gram-negative aerobic and anaerobic bacteria such as, Staphylococci, for example S. aureus and S. epidermidis; Enterococci, for example E. faecium and E. faecalis; Streptococci, for example S. pneumoniae; Haemophilus, for example H. influenzae; Moraxella, for example M. catarrhalis; Escherichia, for example E. coli; Mycobacteria, for example M. tuberculosis and M. ranae; Mycoplasma, for example M. pneumoniae; Pseudomonas, for example P. aeruginosa; intercellular microbes, for example Chlamydia and Rickettsiae. Other examples include Klebsiella pneumoniae, Shigella flexneri, Salmonella typhimurium, Bordetella pertussis, Clostridia perfringens, Helicobacter pylori, Campylobacter jejuni, Legionella pneumophila and Neisseria gonorrhoeae. It is further contemplated that the compounds of the present invention are useful for the treatment of parasitic infections, for example infections caused by Plasmodium falciparum and the like.

Accordingly, in one aspect the present invention relates to a method for the treatment of ailments, the method comprising administering to a subject in need thereof an effective amount of a compound or a composition of this invention. It is contemplated that an effective amount of a compound or a composition of this invention corresponds to an amount of active ingredient, i.e. active compound or a pharmaceutically acceptable salt or ester thereof, in the range of from about 1 μg to about 1000 mg such as, e.g., from about 10 μg to about 500 mg, from about 0.05 to about 100 mg or from about 0.1 to about 50 mg.

In yet another aspect, the present invention relates to use of a compound of this invention for the preparation of a medicament, preferably a medicament for the treatment of infections caused by Gram-positive or Gram-negative aerobic or anaerobic bacteria, or by parasites.

In a preferred embodiment of the invention, there is provided a medicament for the treatment of infections caused by Staphylococci, Enterococci, Streptococci, Haemophilus, Moraxella, Escherichia, Mycobacteria, Mycoplasma, Pseudomonas, Chlamydia, Rickettsia, Klebsiella, Shigella, Salmonella, Bordetella, Clostridia, Helicobacter, Campylobacter, Legionella and Neisseria, preferably caused by Staphylococcus aureus, Staphylococcus epidermidis, Enterococcus faecium, Enterococcus faecalis, Streptococcus pneumoniae, Haemophilus influenzae, Moraxella catarrhalis, Escherichia coli, Mycobacterium tuberculosis, Mycobacterium ranae, Mycoplasma pneumoniae, Pseudomonas aeruginosa, Chlamydia, Rickettsiae, Klebsiella pneumoniae, Shigella flexneri, Salmonella typhimurium, Bordetella pertussis, Clostridia perfringens, Helicobacter pylori, Campylobacter jejuni, Legionella pneumophila and Neisseria gonorrhoeae.

It is further contemplated that the compounds of the present invention are useful for the treatment of parasitic infections, for example infections caused by Plasmodium falciparum and the like.

An intravenous infusion of the compound in 5% dextrose in water or normal saline, or a similar formulation with suitable excipients, is most effective, although an intramuscular bone injection is also useful. Typically, the parenteral dose will be about 0.01 to about 100 mg/kg; preferably between 0.1 and 20 mg/kg, in a manner to maintain the concentration of drug in the plasma at a concentration effective to inhibit PDF. The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent With the condition of the patient. Preferably the oral dose would be about 0.5 to about 50 mg/kg.

No unacceptable toxicological effects are expected when compounds of the present invention are administered in accordance with the present invention.

The compounds of the present invention fully or partly inhibit bacterial PDF, and are thus useful for the treatment and/or prevention of a wide variety of conditions and disorders in which inhibition of PDF is beneficial.

Accordingly, in another aspect the present invention relates to a compound of the general Formula (I) or any optical or geometric isomer or tautomeric form thereof including mixtures of these or a pharmaceutically acceptable salt thereof for use as a pharmaceutical composition.

The invention also relates to pharmaceutical compositions comprising, as an active ingredient, at least one compound of the Formula (I) or any optical or geometric isomer or tautomeric form thereof including mixtures of these or a pharmaceutically acceptable salt thereof together with one or more pharmaceutically acceptable carriers or diluents.

In the following synthetic examples, all of the starting materials were obtained from commercial sources unless otherwise indicated. Without further elaboration, it is believed that one skilled in the art can, using the preceding description, utilize the present invention to its fullest extent. These examples are given to illustrate the invention, not to limit its scope.

### EXAMPLES

Materials and Methods

The starting materials used herein are commercially available or can be prepared according to procedures previously reported in the literature. Unless otherwise stated commercial starting materials were used without further purification. All solvents were HPLC grade. Anhydrous solvents were obtained by storing over 4 Å activated molecular sieves. Synthetic methods to prepare the compounds of this invention might employ protective groups to mask a reactive functionality or minimize unwanted side reactions. Such protective groups are described generally in Green et al. (1999).

Room temperature is approx. 20 degrees centigrade.

Mass spectra (ES-MS spectra) were obtained on a Micromass Quattro micro™ instrument in the positive mode unless otherwise noted.

Materials and Abbreviations
AcOH Acetic acid
BSA Bovine serum albumin
DCM Dichloromethane
DDQ 2,3-Dichloro-5,6-dicyano-p-benzoquinone
DIEA Diisopropylethyl amine
Dhbt-OH 3-Hydroxy-1,2,3-benzotriazin-4(3H)-one
DMF N,N-Dimethyl formamide
DMSO Dimethyl sulfoxide
Fmoc 9-Fluorenylmethoxycarbonyl
HATU O-(7-Azabenzotriazol-1-yl)-N,N,N′,N′tetramethyluroniumhexafluoro-phosphate (from Aldrich; 97%)
HOAt 1-Hydroxy-7-azabenzotriazole (from Aldrich; 98%)
MOPS 4-Morpholinepropanesulfonic acid hemisodium salt
NEM N-Ethyl morpholine (from Fluka; 98%)
POEPOP Polyoxyethylene-polyoxypropylene copolymer
Pf Pentafluorophenyl
RINK 4-[(2,4-Dimethoxyphenyl)-aminomethyl]phenoxyacetic acid
TBTU O-Benzotriazol-1-yl-N,N,N′,N′-tetramethyluronium tetrafluoroborate (from Fluka; 98%)
TFA Trifluoroacetic acid
THF Tetrahydrofurane
TIPS Triisopropyl silane
TMS Trimethylsilyl
TNBS 2,4,6-trinitrobenzene sulfonic acid
Trt Trityl

Piperidine was obtained from Fluka (98%); 3-fluoro-4-nitrobenzoic acid was obtained from Aldrich (98%); SnCl₂ was obtained from Fluka (98%); succinic anhydride was obtained from Aldrich (97%); 3-mercaptopropionic acid was obtained from aldrich (98%); MaOMe was obtained from Aldrich (95%).

All used amines were obtained from Alrich (purity 95-98%).

All used amino acids were obtained from Neosystem (purity 95-99%).

For the biological assay following chemicals were used: Boric acid (Fluka, cat no. 15663); Bovine serum albumin (Fluka, cat no. 5476); Catalase (Fluka, cat no. 60640); f-Met-Ala (Bachem, cat no. G-1855); Methanol (Fluka, cat no. 65544); 4-Morpholine-propanesulfonic acid hemisodium salt (Fluka, cat no. 69947); NaCl (Fluka, cat no. 71382); NaH₂PO₄(Fluka, cat no. 71505); NaOH (Fluka, cat no. 71689); Na₂SO₃ (Fluka, cat no. 71988); 2,4,6-trinitrobenzene sulfonic acid (Fluka, cat no. 92823); sodium 4-(hydroxymercurio)benzoate (Fluka, cat no. 55540).

Synthesis of Compounds of the Invention

Illustrative general methods for the synthesis of the compounds of the invention are illustrated in Scheme A (R₃═—NHCH(R₄)COR₅) and Scheme B (R₃═—NHR₇, —NR₆R₇ or —OR₇) and the individual steps 1-7 are described in Method A and Method B after each scheme, respectively.

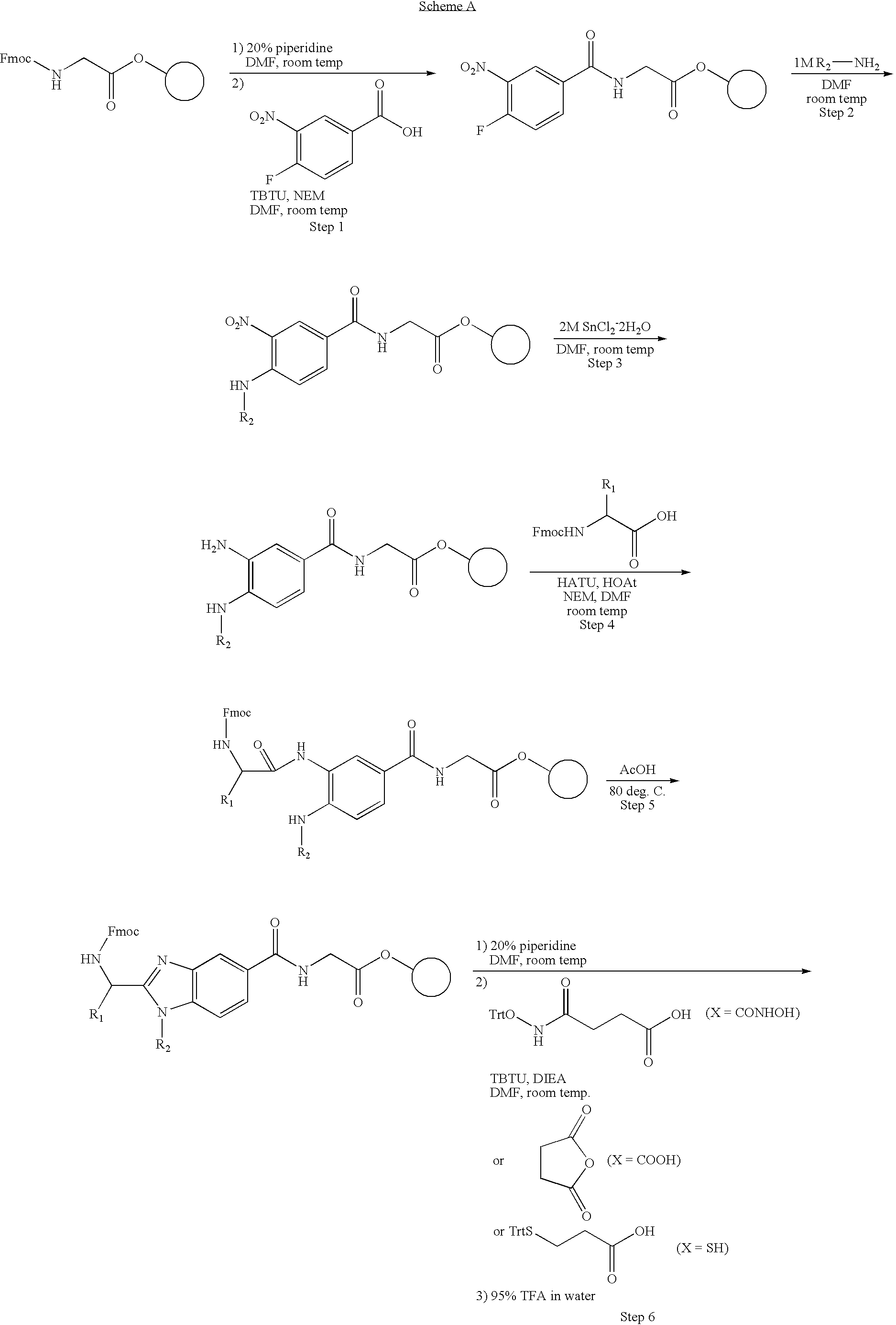

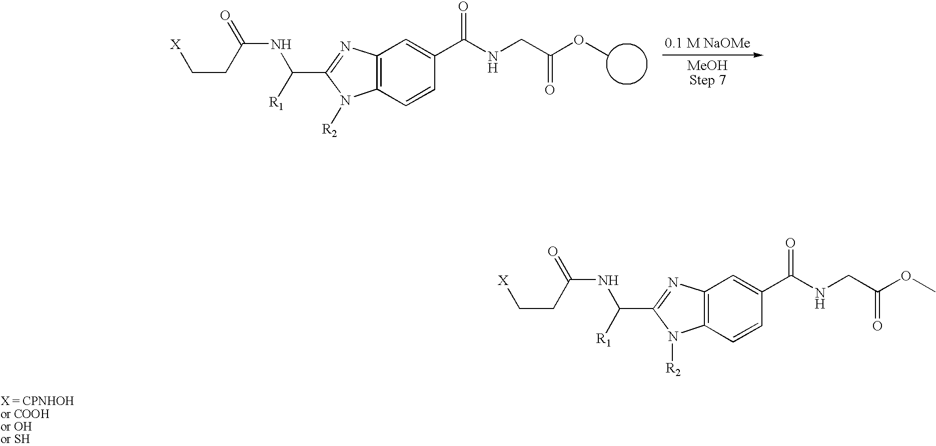

Method A

Step 1:

Resin: Typically 5.0 g of Fmoc protected glycine linked to POEPOP₁₅₀₀-resin (Renil et al. (1996)) (loading˜1.0 mmol/g) was swelled in DMF. Fmoc-deprotection was made by treatment with a 20% solution of piperidine in DMF. A solution of 4-fluoro-3-nitrobenzoic acid (15.0 mmol, 2.8 g, 3 eq.) was dissolved in DMF and activated with TBTU (14.0 mmol, 4.50 g, 2.8 eq) and NEM (20.0 mmol, 2.30 g, 4 eq). The acid was activated for 15 min prior to being added to the swelled resin. Reaction was run over night at room temperature. The resin was then rinsed with DMF (10 times), DCM (10 times) and finally MeCN (10 times). The resin was lyophilized.

Step 2:

Dry resin (typically 800 mg, approx. 0.5 mmol) was placed in a plastic syringe and washed 5 times with DMF. To each resin a 10% solution (in DMF) of appropriate amine was added. After 3 hours the resins were washed with DMF (10 times) and DMSO (3 times) and used directly in step 3.

Step 3:

To each resin was added a 2M solution of SnCl₂ in DMF. The resin mixture was allowed to stand at room temperature over night before removal of the liquid phase. The resin was washed With DMF (10 times) and used directly in step 4.

Step 4:

A solution of HATU (0.95 mmol, 361 mg), HOAt (0.35 mmol, 50 mg), NEM (1.0 mmol, 126 μl) and the appropriate Fmoc-protected aminoacid in DMF was added to the resin and allowed to react for 48 h at room temperature. The resin was washed with DMF (10 times) and used directly in step 5.

Step 5:

The resin was washed with AcOH (3 times) and finally AcOH was added to the resin and warmed to 80 degrees C. for 48 h. The resin was then washed with DMF (10 times) and used directly in the next step.

Step 6:

The resin was treated with 20% piperidine in DMF for 30 min at room temperature and then rinsed with DMF (8 times).

(X═CONHOH): O-Tritylsuccinamic acid (3.7 equiv) was dissolved in DMF and activated with TBTU (3.0 equiv) and NEM (3.7). The TBTU activated succinamic acid was added to the resin and reacted over night. The resin was thereafter rinsed with DMF (10 times) and then treated with 95% TFA in DMF to remove the trityl protecting group.

(X═COOH, OH): Succinic anhydride (10 equiv) was dissolved in DMF and added to the resin. The reaction was allowed to proceed at room temperature for 60 minutes at room temperature. The resin was rinsed with DMF (8 times) and then treated with 95% TFA in water.

(X═SH): S-trityl protected mercaptopropionic acid (4 equiv) was dissolved in DMF (into a concentration of 0.1 M) and activated with TBTU (3.5 equiv) and NEM (4 equiv). The activated propionic acid was then added to the resin and the reaction was allowed to proceed over night. The resin was washed with DMF (10 times) and then treated with 95% TFA in DMF to cleave off the trityl protecting group.

Step 7:

The desired benzimidazoles were cleaved off the resin by treatment of 0.1M NaOMe in methanol. The products were collected and lyophilized. Analyses of the products were made by LC-MS (Liquid Chromatography-Mass Spectrometry).

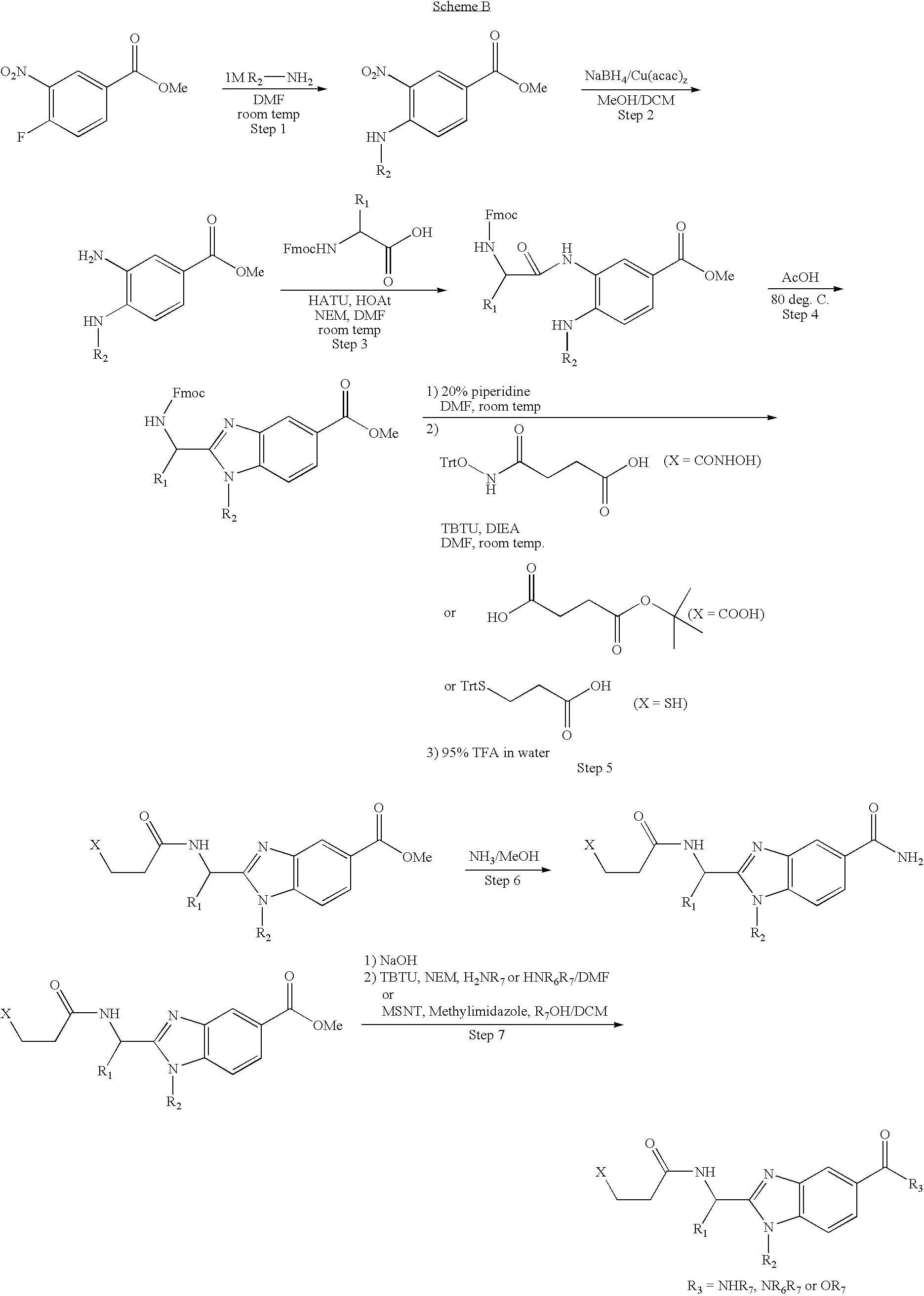

Method B:

Step 1:

Typically 1 mmol of 4-fluoro-3-nitrobenzoic acid methylester was placed in a plastic tube and dissolved in THF (5 ml). To this solution was added 2 mmol of amine (in a 1 M solution in THF). To this solution was added triethylamine (2 ml). The solution was stirred at room temperature over night. The solvent was removed in vacuo and the residue was passed through a short silica plug.

Step 2:

The product from step 1 (typically 1 mmol) was dissolved in DCM/MeOH (50:50, typically 3 ml). In a separate MeOH solution (typically 20 ml) NaBH₄ (10 equiv.) and Cu(acac)₂ (0.6 equiv.). To the borohydride/Cu(acac)₂ solution was added the product from step 1. The reaction mixture was stirred at room temperature for 2 h. The solvent was removed. Water was added and the aqueous solution was extracted with EtOAc. Drying and removal of the solvent yields the desired product used directly in the next step.

Step 3:

Typically a solution of HATU (0.95 mmol, 361 mg), HOAt (0.35 mmol, 50 mg), NEM (1.0 mmol, 126 μl) and the appropriate Fmoc-protected aminoacid in DMF was added to a solution of the product from step 2. The reaction was run over night at room temperature. The solvent was removed in vacuo. Water was added and extracted with EtOAc. Drying and removal of the solvent yields the product, which is purified by column chromatography.

Step 4:

The product from step 3 was dissolved in AcOH and warmed to 60 C over night. The solvent was removed in vacuo and the product used directly in the next reaction step.

Step 5:

The product from step 4 was treated with 20% piperidine in DMF for 30 min at room temperature. The solvent was removed in vacuo, and was then alternatively treated with:

(X═CONHOH): O-Tritylsuccinamic acid (3.7 equiv) was dissolved in DMF and activated with TBTU (3.0 equiv) and NEM (3.7). The TBTU activated succinamic acid was added to the resin and reacted over night. The solvent was removed in vacuo and water was then added. The aqueous phase was extracted with EtOAc. Drying and removal of the solvent gave the crude product which was then treated with 95% TFA in DCM to remove the trityl protecting group. The product was purified by prep. HPLC.

(X═COOH, OH): Mono-tert-butyl succinate (10 equiv) was dissolved in THF and added to a solution in THF of the product from step 4. The reaction was allowed to proceed at room temperature for 60 minutes at room temperature. The solvent was removed in vacuo and water was then added. The aqueous phase was extracted with EtOAc. Drying and removal of the solvent gave the crude product which was then treated with 95% TFA in DCM to remove the tert-butyl group. The product was purified by prep. HPLC.

(X═SH): S-trityl protected mercaptopropionic acid (4 equiv) was dissolved in DMF (into a concentration of 0.1 M) and activated with TBTU (3.5 equiv) and NEM (4 equiv). The activated propionic acid was then added to the product from step 4 and was allowed to proceed over night. The solvent was removed in vacuo and water was then added. The aqueous phase was extracted with EtOAc. Drying and removal of the solvent gave the crude product which was then treated with 95% TFA in DCM to remove the trityl protecting group. The product was purified by prep. HPLC.

Step 6:

The respective product from step 5 were dissolved in NH₃/MeOH (7M) and stirred at room temp. over night. The solvent was removed in vacuo and the product purified by prep. HPLC.

Step 7:

The respective product from step 5, omitting the usage of TFA and purification the crude products, were dissolved in MeOH and 1 equiv. of NaOH (1M solution in MeOH) was added. Reaction was run at room temp. over night. The solvent was then removed.

(R₃═NHR₇, NR₆R₇) the hydrolysed ester (1 equiv.) was dissolved in DMF and activated with TBTU (1 equiv.) and NEM (1 equiv.). The respective amine (1.2 equiv.; NH₂R₇ or NHR₆R₇) dissolved in DMF was then added to the activated acid. The reaction was run over night. The solvent was removed in vacuo and the crude product was dissolved in TFA and purified by prep. HPLC.

(R₃═OR₇) the hydrolysed ester (1 equiv.) was dissolved in DMF and activated with MSNT (1 equiv.) and methylimidazole (1 equiv.). The respective alcohol (1.2 equiv.; R₇OH) dissolved in DMF was then added to the activated acid. The reaction was run over night. The solvent was removed in vacuo and the crude product was dissolved in TFA and purified by prep. HPLC.

Biological Assays

The compounds of this invention may be tested in the following biological assay in order to determine the concentration of compound (IC₅₀) required for exhibiting the desired pharmacological effect.

To find inhibitors of Peptide Deformylase (PDF), a colorimetric cell free assay for measuring the enzymatic activity of PDF has been adapted to the microtiter plate format (96 wells). The assay comprises three components, purified PDF, f-Met-Ala as substrate and TNBS as the detecting agent of primary amino groups. The resulting TNP—NH-Met-Ala sulfite complex can be detected at 420 nm. PDF enzymes, containing Fe²⁺ as the native metal, are purified and are stabilized by the addition of tris(2-carboxyethyl)phosphine (TCEP).

Bacterial Peptide Deformylase (PDF) Assay

The IC₅₀ value of a compound of the invention as a bacterial PDF inhibitor was determined using the following assay.

Materials:

Assay buffer 0.1 M MOPS pH was adjusted to 7.2 with NaOH, containing 0.25 M NaCl, 100 μg/mL catalase and 1 mg/mL BSA.

Enzyme Mix:

E. coli enzyme (2.5 mg/ml) 10 μl+290 μl Assay buffer, 1 μl per ml enzyme mix. S. aureus (15 mg/ml) 10 μl+990 μl Assay buffer, 0.3 μl per ml enzyme mix.

Substrate mix: 10 mM f-Met-Ala was made up from 200 mM f-Met-Ala in methanol with assay buffer.

TNBS solution: Freshly dilute 1 M TNBS stock solution diluted 1:10 with water.

Buffer C: 0.5 M borate buffer adjusted to pH 9.5 with NaOH.

Buffer D: 0.2 ml of freshly prepared 0.5 M Na₂SO₃ was mixed with 9.8 mL of 0.5M NaH₂PO₄.

Inhibitor solution: 2 mM Sodium 4-(hydroxymercurio)benzoate in assay buffer.

Compound mix: Compound of formula I dissolved in DMSO in a 10 mg/mL stock solution. Further dilutions were made in DMSO in the concentration range between 0.05 to 100 mM.

Method (Assay Conditions):

The assay was performed in a 96 Microtiter plate containing test compound. To each well containing test compound mix was added 75 microliter of enzyme mix from E. coli followed by the addition of 25 microliter of substrate mix. The resulting mix was incubated for 30 minutes at room temperature with shaking. TNBS solution (50 microliter/well) was added and the resulting mixture was incubated for 15 minutes under shaking. Buffer C was then added (20 microliter/well). After incubating at room temperature for 15 minutes under shaking, buffer D was added (50 microliter/well). The optical diffraction was then measured at 420 nm, thereby determining the IC₅₀ value.

The assay was repeated using enzyme mix from S. aureus.

The compounds and processes of the invention will be better understood-in connection with the following examples, which are intended as an illustration of and not as a limitation upon the scope of the invention.

### Example 1

### ({1-Cyclopropyl-2-[1-(3-mercapto-propionylamino)-propyl]-1H-benzoimidazole-5-carbonyl}-amino)-acetic acid methyl ester

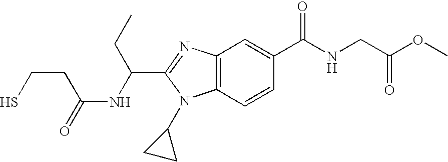

The title compound was prepared according to Method A using cyclopropyl amine in step 2, alpha-aminobutanoic acid in step 4 and S-trityl protected mercaptopropionic acid in step 6.

Mass found: 418.919.

IC₅₀ (microM): 65.0 (enzyme from E. coli)
45.4 (enzyme from S. aureus).

### Example 2

### ({1-(4-Chloro-benzyl)-2-[1-(3-mercapto-propionylamino)-2-phenyl-ethyl]-1H-benzoimidazole-5-carbonyl}-amino)-acetic acid methyl ester

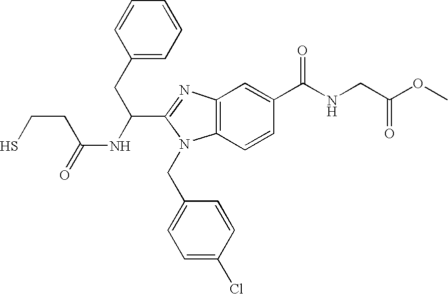

The title compound was prepared according to Method A using 4-chlorobenzyl amine in step 2, phenylalanine acid in step 4 and S-trityl protected mercaptopropionic acid in step 6.

Mass found: 564.774.

IC₅₀ (microM): 22.1 (enzyme from E. coli)
6.1 (enzyme from S. aureus).

### Example 3

### N-{1-[1-Benzyl-5-(methoxycarbonylmethyl-carbamoyl)-1H-benzoimidazol-2-yl]-3-methylsulfanyl-propyl}-succinamic acid

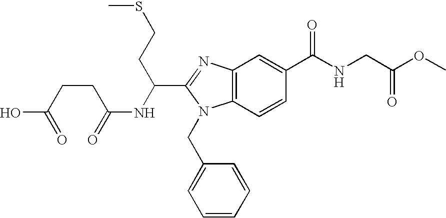

The title compound was prepared according to Method A using benzyl amine in step 2, methionen in step 4 and succinic acid anhydride in step 6.

Mass found: 526.863.

IC₅₀ (microM): 80.0 (enzyme from E coli)
31.4 (enzyme from S. aureus).

### Example 4

### N-{1-[1-Butyl-5-(methoxycarbonylmethyl-carbamoyl)-1H-benzoimidazol-2-yl]-2-phenyl-ethyl}-succinamic acid

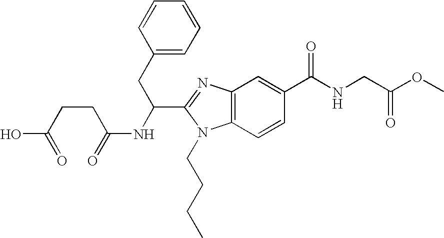

The title compound was prepared according to Method A using butyl amine in step 2, phenylalanine in step 4 and succinic acid anhydride in step 6.

Mass found: 508.899.

IC₅₀ (microM): 2.8 (enzyme from E. coli)
1.0 (enzyme from S. aureus).

### Example 5

### N-{1-[1-Furan-2-ylmethyl-5-(methoxycarbonylmethyl-carbamoyl)-1H-benzoimidazol-2-yl]-2-phenyl-ethyl}-succinamic acid

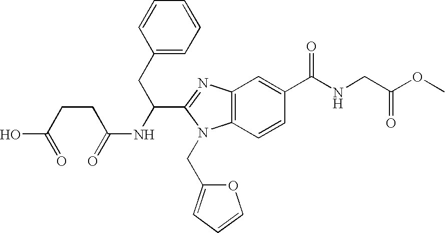

The title compound was prepared according to Method A using furylmethyl amine in step 2, phenylalanine in step 4 and succinic acid anhydride in step 6.

Mass found: 532.875.

IC₅₀ (microM): 12.9 (enzyme from E. coli)
1.6 (enzyme from S. aureus).

### Example 6

### N-{1-[1-(4-Chloro-benzyl)-5-(methoxycarbonylmethyl-carbamoyl)-1H-benzoimidazol-2-yl]-2-phenyl-ethyl}-succinamic acid

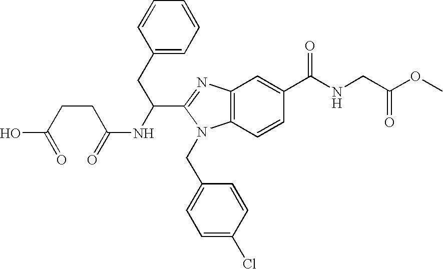

The title compound was prepared according to Method A using 4-chlorobenzyl amine in step 2, phenylalanine in step 4 and succinic acid anhydride in step 6.

Mass found: 576.825.

IC₅₀ (microM): 1.3 (enzyme from E. coli)
0.093 (enzyme from S. aureus).

### Example 7

### N-{1-[1-Cyclopropyl-5-(methoxycarbonylmethyl-carbamoyl)-1H-benzoimidazol-2-yl]-3-phenyl-propyl}-succinamic acid

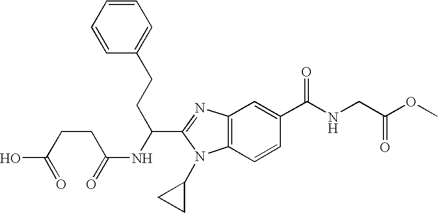

The title compound was prepared according to Method A using cyclopropyl amine in step 2, homophenylalanine in step 4 and succinic acid anhydride in step 6.

Mass found: 506.884.

IC₅₀ (microM): 2.6 (enzyme from E. coli)
2.7 (enzyme from S. aureus).

### Example 8

### N-{1-[1-Cyclohexylmethyl-5-(methoxycarbonylmethyl-carbamoyl)-1H-benzoimidazol-2-yl]-3-phenyl-propyl}-succinamic acid

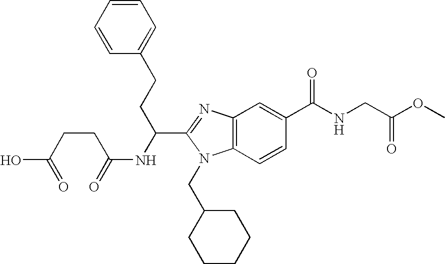

The title compound was prepared according to Method A using cyclohexylmethyl amine in step 2, homophenylalanine in step 4 and succinic acid anhydride in step 6.

Mass found: 562.930.

IC₅₀ (microM): 29.8 (enzyme from E. coli)
15.1 (enzyme from S. aureus).

### Example 9

### N-{1-[1-(2-Chloro-benzyl)-5-(methoxycarbonylmethyl-carbamoyl)-1H-benzoimidazol-2-yl]-3-phenyl-propyl}-succinamic acid

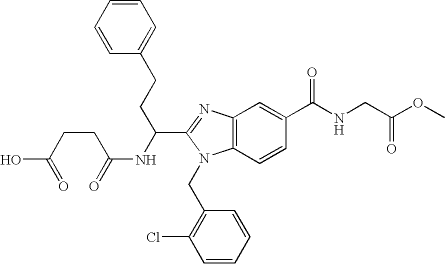

The title compound was prepared according to Method A using 2-chlorobenzyl amine in step 2, homophenylalanine in step 4 and succinic acid anhydride in step 6.

Mass found: 590.821.

IC₅₀ (microM): 27.8 (enzyme from E. coli)
18.0 (enzyme from S. aureus).

### Example 10

### N-{1-[1-Cyclopropyl-5-(methoxycarbonylmethyl-carbamoyl)-1H-benzoimidazol-2-yl]-propyl}-succinamic acid

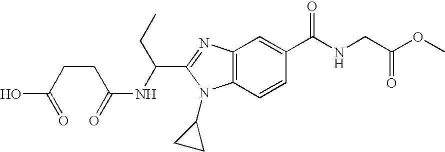

The title compound was prepared according to Method A using cyclopropyl amine in step 2, alpha-aminobutyric acid in step 4 and succinic acid anhydride in step 6.

Mass found: 430.926.

IC₅₀ (microM): 5.6 (enzyme from E. coli)
0.023 (enzyme from S. aureus).

### Example 11

### N-{1-[1-Furan-2-ylmethyl-5-(methoxycarbonylmethyl-carbamoyl)-1H-benzoimidazol-2-yl]-propyl}-succinamic acid

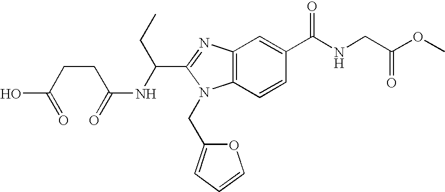

The title compound was prepared according to Method A using furylmethyl amine in step 2, alpha-aminobutyric acid in step 4 and succinic acid anhydride in step 6.

Mass found: 470.877.

IC₅₀(microM): 2.8 (enzyme from E. coli)
1.4 (enzyme from S. aureus).

### Example 12

### N-{1-[1-Benzyl-5-(methoxycarbonylmethyl-carbamoyl)-1H-benzoimidazol-2-yl]-propyl}-succinamic acid

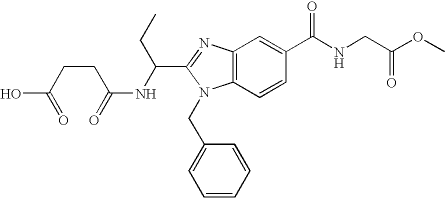

The title compound was prepared according to Method A using benzyl amine in step 2, alpha-aminobutyric acid in step 4 and succinic acid anhydride in step 6.

Mass found: 480.906.

IC₅₀ (microM): 4.8 (enzyme from E. coli)
0.068 (enzyme from S. aureus).

### Example 13

### N-{1-[1-Cyclopropyl-5-(methoxycarbonylmethyl-carbamoyl)-1H-benzoimidazol-2-yl]-3-methyl-butyl}-succinamic acid

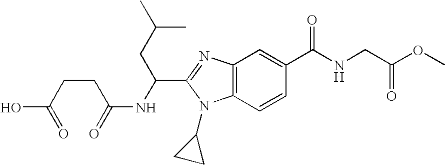

The title compound was prepared according to Method A using cyclopropyl amine in step 2, leucine in step 4 and succinic acid anhydride in step 6.

Mass found: 459.932.

IC₅₀ (microM): 4.0 (enzyme from E. coli)
0.119 (enzyme from S. aureus).

### Example 14

### N-{1-[1-Butyl-5-(methoxycarbonylmethyl-carbamoyl)-1H-benzoimidazol-2-yl]-3-methyl-butyl}-succinamic acid

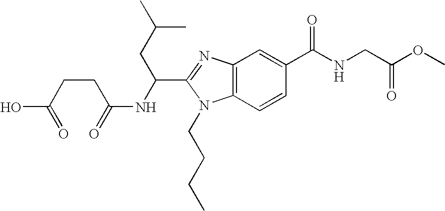

The title compound was prepared according to Method A using butyl amine in step 2, leucine in step 4 and succinic acid anhydride in step 6.

Mass found: 474.962.

IC₅₀ (microM): 7.3 (enzyme from E. coli)
2.6 (enzyme from S. aureus).

### Example 15

### N-{1-[1-Benzyl-5-(methoxycarbonylmethyl-carbamoyl)-1H-benzoimidazol-2-yl]-3-methyl-butyl}-succinamic acid

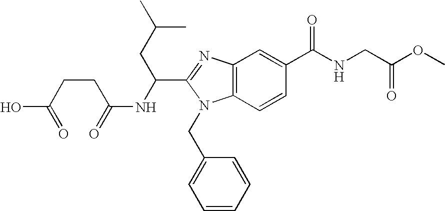

The title compound was prepared according to Method A using benzyl amine in step 2, leucine in step 4 and succinic acid anhydride in step 6.

Mass found: 508.910.

IC₅₀ (microM): 3.3 (enzyme from E. coli)
4.0 (enzyme from S. aureus).

### Example 16

### N-{1-[1-Cyclopropyl-5-(methoxycarbonylmethyl-carbamoyl)-1H-benzoimidazol-2-yl]-3-methylsulfanyl-propyl}-succinamic acid

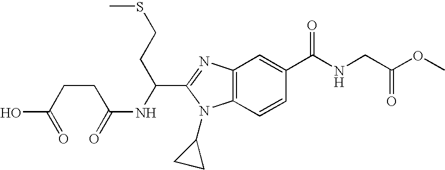

The title compound was prepared according to Method A using cyclopropyl amine in step 2, methionine in step 4 and succinic acid anhydride in step 6.

Mass found: 476.897.

IC₅₀ (microM): 47.2 (enzyme from E. coli)
17.6 (enzyme from S. aureus).

### Example 17

### N-{1-[5-(Methoxycarbonylmethyl-carbamoyl)-1-(2-thiophen-2-yl-ethyl)-1H-benzoimidazol-2-yl]-2-naphthalen-1-yl-ethyl}-succinamic acid

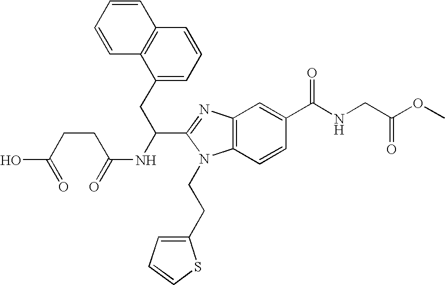

The title compound was prepared according to Method A using thiophenetyl amine in step 2, 1-naphthyl alanine in step 4 and succinic acid anhydride in step 6.

Mass found: 612.849.

IC₅₀ (microM): 0.071 (enzyme from E. coli)
0.099 (enzyme from S. aureus).

### Example 18

### N-{1-[1-Butyl-5-(methoxycarbonylmethyl-carbamoyl-1H-benzoimidazol-2-yl-2-naphthalen-1-yl-ethyl}-succinamic acid

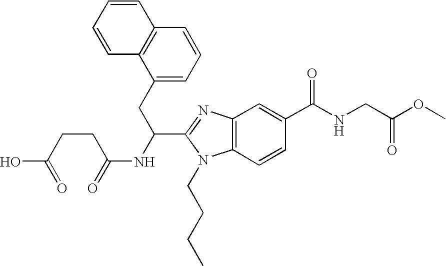

The title compound was prepared according to Method A using butyl amine in step 2, 1-naphthyl alanine in step 4 and succinic acid anhydride in step 6.

Mass found: 558.894.

IC₅₀ (microM): 2.7 (enzyme from E. coli)
0.074 (enzyme from S. aureus).

### Example 19

### ({2-[5-Amino-1-(3-mercapto-propionylamino)-pentyl]-1-cyclohexylmethyl-1H-benzoimidazole-5-carbonyl}-amino)-acetic acid methyl ester

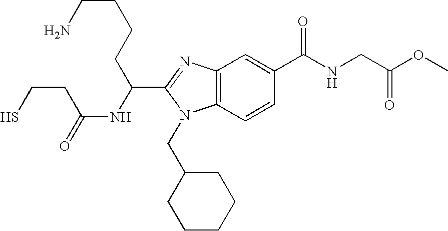

The title compound was prepared according to Method A using cyclohexylmethyl amine in step 2, Boc-lysine in step 4 and S-trityl protected mercaptopropionic acid in step 6.

Mass found: 518.218.

IC₅₀ (microM): 38.1 (enzyme from E. coli)
27.1 (enzyme from S. aureus).

### Example 20

### ({1-Cyclopropyl-2-[2-(4-hydroxy-phenyl)-1-(3-mercapto-propionylamino)-ethyl]-1H-benzoimidazole-5-carbonyl}-amino)-acetic acid methyl ester

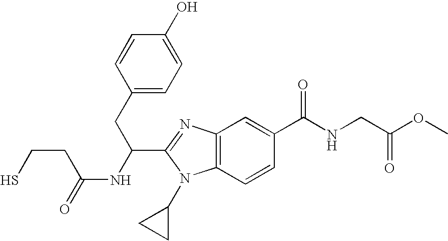

The title compound was prepared according to Method A using cyclopropyl amine in step 2, tyrosine in step 4 and S-trityl protected mercaptopropionic acid in step 6.

Mass found: 497.108.

IC₅₀ (microM): >200 (enzyme from E. coli)
>200 (enzyme from S. aureus).

### Example 21

### ({1-Cyclohexylmethyl-2-[2-(4-hydroxy-phenyl)-1-(3-mercapto-propionylamino)-ethyl]-1H-benzoimidazole-5-carbonyl}-amino)-acetic acid methyl ester

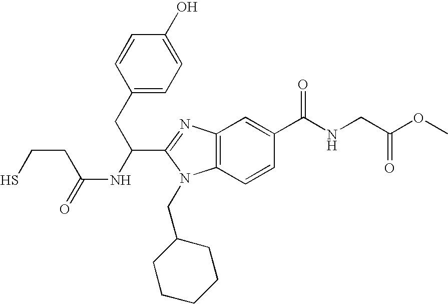

The title compound was prepared according to Method A using cyclohexylmethyl amine in step 2, tyrosine in step 4 and S-trityl protected mercaptopropionic acid in step 6.

Mass found: 553.146.

IC₅₀ (microM): >200 (enzyme from E. coli)
>200 (enzyme from S. aureus).

### Example 22

### ({1-(2-Hydroxy-ethyl)-2-[1-(3-mercapto-propionylamino)-2-phenyl-ethyl]-1H-benzoimidazole-5-carbonyl}-amino)-acetic acid methyl ester

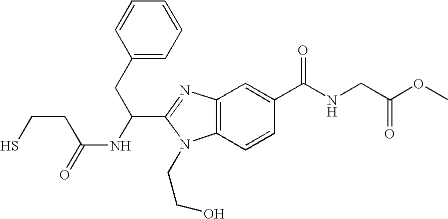

The title compound was prepared according to Method A using aminoethanol in step 2, phenyl alanine in step 4 and S-trityl protected mercaptopropionic acid in step 6.

Mass found: 485.133.

IC₅₀ (microM): >200 (enzyme from E. coli)
>200 (enzyme from S. aureus).

### Example 23

### N-{5-Amino-1-[1-cyclopropyl-5-(methoxycarbonylmethyl-carbamoyl)-1H-benzoimidazol-2-yl-pentyl}-succinamic acid

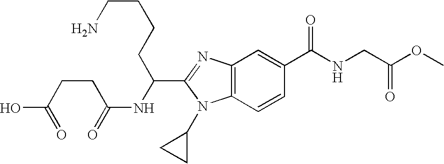

The title compound was prepared according to Method A using cyclopropyl amine in step 2, Boc-lysine in step 4 and succinicacid anhydride in step 6.

Mass found: 474.204.

IC₅₀ (microM): 0.3 (enzyme from E. coli)
0.3 (enzyme from S. aureus).

### Example 24

### N-{5-Amino-1-[1-cyclohexylmethyl-5-(methoxycarbonylmethyl-carbamoyl)-1H-benzoimidazol-2-yl]-pentyl}-succinamic acid

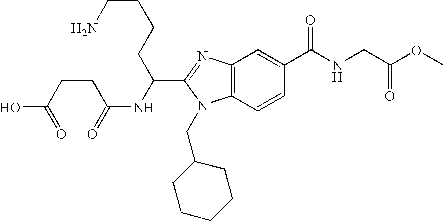

The title compound was prepared according to Method A using cyclohexylmethyl amine in step 2, Boc-lysine in step 4 and succinicacid anhydride in step 6.

Mass found: 530.225.

IC₅₀ (microM): 13.1 (enzyme from E. coli)
1.4 (enzyme from S. aureus).

### Example 25

### N-[1-[1-Cyclopropyl-5-(methoxycarbonylmethyl-carbamoyl)-1H-benzoimidazol-2-yl]-2-(4-hydroxyphenyl)-ethyl]-succinamic acid

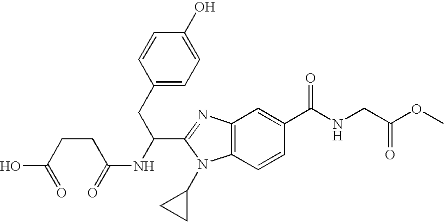

The title compound was prepared according to Method A using cyclopropyl amine in step 2, tyrosine in step 4 and succinicacid anhydride in step 6.

Mass found: 509.143.

IC₅₀ (microM): 12.8 (enzyme from E. coli)
28.3 (enzyme from S. aureus).

### Example 26

### N-[1-[1-Cyclohexylmethyl-5-(methoxycarbonylmethyl-carbamoyl)-1H-benzoimidazol-2-yl]-2-(4-hydroxyphenyl)-ethyl]-succinamic acid

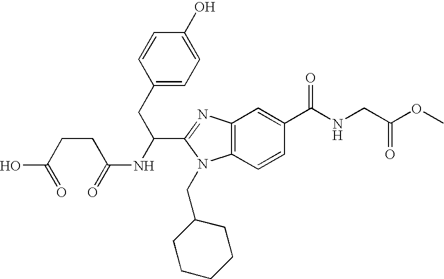

The title compound was prepared according to Method A using cyclohexylmethyl amine in step 2, tyrosine in step 4 and succinicacid anhydride in step 6.

Mass found: 565.182.

IC₅₀(microM): 35.2 (enzyme from E. coli)
25.0 (enzyme from S. aureus).

### Example 27

### N-{1-[1-(2-Hydroxy-ethyl)-5-(methoxycarbonylmethyl-carbamoyl)-1H-benzoimidazol-2-yl]-2-phenyl-ethyl}-succinamic acid

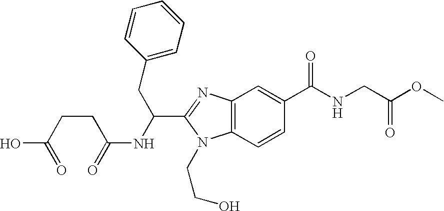

The title compound was prepared according to Method A using aminoethanol in step 2, phenylalanine in step 4 and succinicacid anhydride in step 6.

Mass found: 497.155.

IC₅₀ (microM): 41.8 (enzyme from E. coli)
27.7 (enzyme from S. aureus).

### Example 28

### N-{1-[1-(2-Hydroxy-ethyl)-5-(methoxycarbonylmethyl-carbamoyl)-1H-benzoimidazol-2-yl]-3-methylbutyl}-succinamic acid

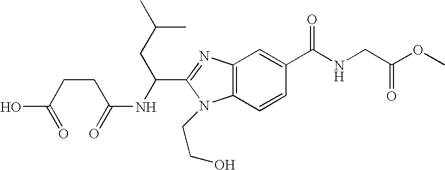

The title compound was prepared according to Method A using aminoethanol in step 2, leucine in step 4 and succinicacid anhydride in step 6.

Mass found: 463.176.

IC₅₀ (microM): >200 (enzyme from E. coli)
34.4 (enzyme from S. aureus).

### Example 29

### ({1-Cyclopropyl-2-[1-(3-hydroxycarbamoyl-propionylamino)-propyl]-1H-benzoimidazole-5-carbonyl}-amino)-acetic acid methyl ester

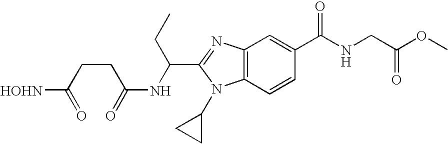

The title compound was prepared according to Method A using cyclopropyl amine in step 2, alpha-aminobutyric acid in step 4 and O-trityl succinicacid hydroxamide in step 6.

Mass found: 446.115.

IC₅₀ (microM): 0.079 (enzyme from E. coli)
0.105 (enzyme from S. aureus).

### Example 30

### N-{1-[1-Benzyl-5-(methoxycarbonylmethyl-carbamoyl)-1H-benzoimidazol-2-yl]-2-naphthalen-1-yl-ethyl}-succinamic acid

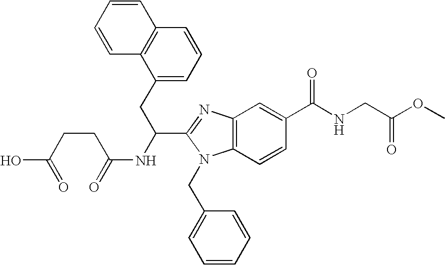

The title compound was prepared according to Method A using benzyl amine in step 2, 1-naphthyl alanine in step 4 and succinic acid anhydride in step 6.

Mass found: 592.864.

IC₅₀ (microM): 7.0 (enzyme from E. coli)
0.3 (enzyme from S. aureus).

### Example 31

### ({1-Furan-2-ylmethyl-2-[1-(3-hydroxycarbamoyl-propionylamino)-propyl]-1H-benzoimidazole-5-carbonyl}-amino)-acetic acid methyl ester

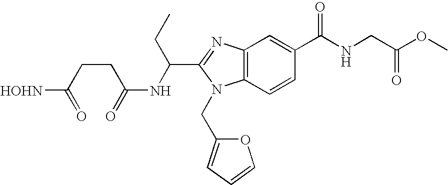

The title compound was prepared according to Method A using furylmethyl amine in step 2, alpha-aminobutyric acid in step 4 and O-trityl succinicacid hydroxamide in step 6.

Mass found: 486.090.

IC₅₀ (microM): 0.098 (enzyme from E. coli)
0.151 (enzyme from S. aureus).

### Example 32

### ({1-Benzyl-2-[1-(3-hydroxycarbamoyl-propionylamino)-propyl]-1H-benzoimidazole-5-carbonyl}-amino)-acetic acid methyl ester

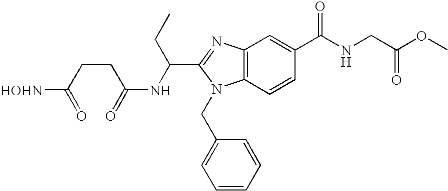

The title compound was prepared according to Method A using benzyl amine amine in step 2, alpha-aminobutyric acid in step 4 and O-trityl succinicacid hydroxamide in step 6.

Mass found: 496.112.

IC₅₀ (microM): 0.045 (enzyme from E. coli)
0.067 (enzyme from S. aureus).

### Example 33

### ({1-Cyclopropyl-2-[1-(3-hydroxycarbamoyl-propionylamino)-3-methyl-butyl]-1H-benzoimidazole-5-carbonyl}-amino)-acetic acid methyl ester

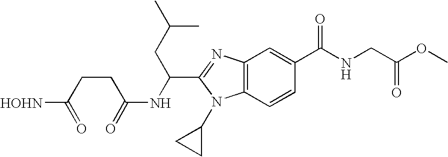

The title compound was prepared according to Method A using cyclopropyl amine amine in step 2, leucine in step 4 and O-trityl succinicacid hydroxamide in step 6.

Mass found: 474.123.

IC₅₀ (microM): 0.3 (enzyme from E. coli)
0.496 (enzyme from S. aureus).

### Example 34

### ({1-Butyl-2-[1-(3-hydroxycarbamoyl-propionylamino)-3-methyl-butyl]-1H-benzoimidazole-5-carbonyl}-amino)-acetic acid methyl ester

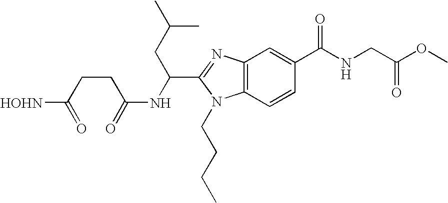

The title compound was prepared according to Method A using butyl amine amine in step 2, leucine in step 4 and O-trityl succinicacid hydroxamide in step 6.

Mass found: 490.140.

IC₅₀ (microM): 0.3 (enzyme from E coli)
2.266 (enzyme from S. aureus).

### Example 35

### ({1-Benzyl-2-[1-(3-hydroxycarbamoyl-propionylamino)-3-methyl-butyl]-1H-benzoimidazole-5-carbonyl}-amino)-acetic acid methyl ester

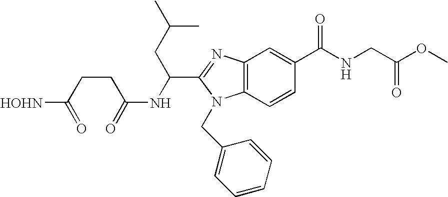

The title compound was prepared according to Method A using benzyl amine amine in step 2, leucine in step 4 and O-trityl succinicacid hydroxamide in step 6.

Mass found: 524.104.

IC₅₀ (microM): 0.3 (enzyme from E. coli)
0.804 (enzyme from S. aureus).

### Example 36

### ({1-Cyclopropyl-2-[1-(3-hydroxycarbamoyl-propionylamino)-3-methylsulfanyl-propyl]-1H-benzoimidazole-5-carbonyl}-amino)-acetic acid methyl ester

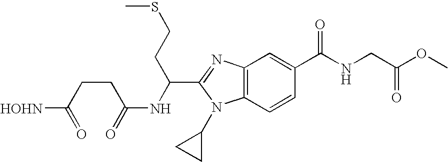

The title compound was prepared according to Method A using cyclopropyl amine amine in step 2, methionine in step 4 and O-trityl succinicacid hydroxamide in step 6.

Mass found: 508.057 (only the sulfoxide could be detected).

IC₅₀ (microM): 0.3 (enzyme from E. coli)
1.5 (enzyme from S. aureus).

### Example 37

### ({1-Cyclohexylmethyl-2-[1-(3-hydroxycarbamoyl-propionylamino)-3-methylsulfanyl-propyl]-1H-benzoimidazole-5-carbonyl}-amino)-acetic acid methyl ester

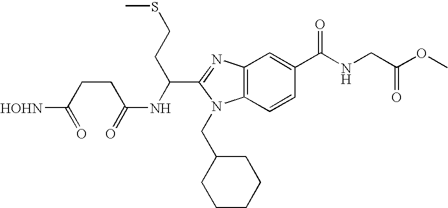

The title compound was prepared according to Method A using cyclohexylmethyl amine amine in step 2, methionine in step 4 and-O-trityl succinicacid hydroxamide in step 6.

Mass found: 564.577 (only the sulfoxide could be detected).

IC₅₀ (microM): 0.317 (enzyme from E. coli)
0.3 (enzyme from S. aureus).

### Example 38

### (1-Benzyl-2-[1-(3-hydroxycarbamoyl-propionylamino)-3-methylsulfanyl-propyl]-1H-benzoimidazole-5-carbonyl}-amino)-acetic acid methyl ester

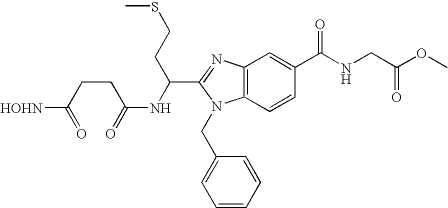

The title compound was prepared according to Method A using benzyl amine in step 2, methionine in step 4 and O-trityl succinicacid hydroxamide in step 6.

Mass found: 558.040 (only the sulfoxide could be detected).

IC₅₀ (microM): 25.8 (enzyme from E. coli)
3.5 (enzyme from S. aureus).

### Example 39

### ({1-Furan-2-ylmethyl-2-[1-(3-hydroxycarbamoyl-propionylamino)-2-phenyl-ethyl]-1H-benzoimidazole-5-carbonyl}-amino)-acetic acid methyl ester

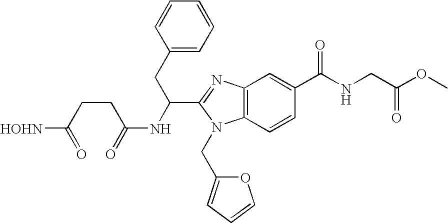

The title compound was prepared according to Method A using furylmethyl amine in step 2, phenylalanine in step 4 and O-trityl succinicacid hydroxamide in step 6.

Mass found: 548.059.

IC₅₀ (microM): 1.5 (enzyme from E. coli)
0.3 (enzyme from S. aureus).

### Example 40

### ({1-(4-Chloro-benzyl)-2-[1-(3-hydroxycarbamoyl-propionylamino)-2-phenyl-ethyl]-1H-benzoimidazole-5-carbonyl}-amino)-acetic acid methyl ester

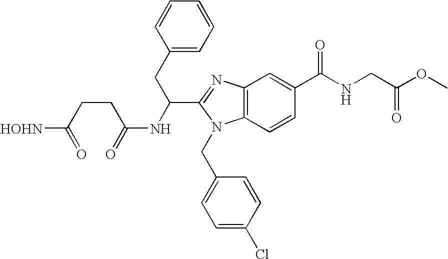

The title compound was prepared according to Method A using 4-chlorobenzyl amine in step 2, phenylalanine in step 4 and O-trityl succinicacid hydroxamide in step 6.

Mass found: 592.044.

IC₅₀ (microM): 1.4 (enzyme from E. coli)
0.021 (enzyme from S. aureus).

### Example 41

### ({1-Cyclopropyl-2-[1-(3-hydroxycarbamoyl-propionylamino)-3-phenyl-propyl]-1H-benzoimidazole-5-carbonyl}-amino)-acetic acid methyl ester

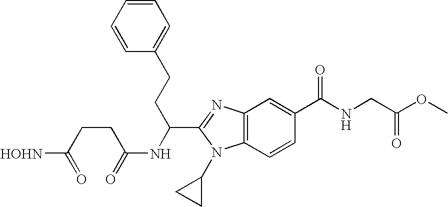

The title compound was prepared according to Method A using cyclopropyl amine in step 2, homophenylalanine in step 4 and O-trityl succinicacid hydroxamide in step 6.

Mass found: 522.095.

IC₅₀ (microM): 2.3 (enzyme from E. coli)
0.363 (enzyme from S. aureus).

### Example 42

### ({1-Cyclohexylmethyl-2-[1-(3-hydroxycarbamoyl-propionylamino)-3-phenyl-propyl]-1H-benzoimidazole-5-carbonyl}-amino)-acetic acid methyl ester

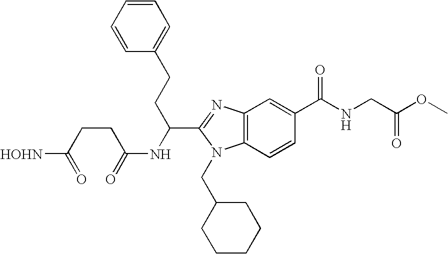

The title compound was prepared according to Method A using cyclohexylmethyl amine in step 2, homophenylalanine in step 4 and O-trityl succinicacid hydroxamide in step 6.

Mass found: 578.138.

IC₅₀ (microM): 0.3 (enzyme from E. coli)
0.3 (enzyme from S. aureus).

### Example 43

### ({1-(2-Chloro-benzyl)-2-[1-(3-hydroxycarbamoyl-propionylamino)-2-phenyl-ethyl]-1H-benzoimidazole-5-carbonyl}-amino)-acetic acid methyl ester

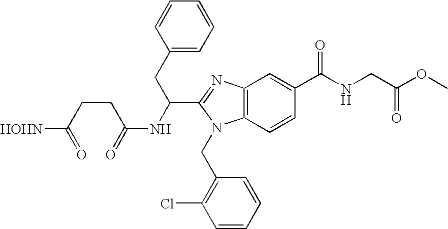

The title compound was prepared according to Method A using 2-chlorobenzyl amine in step 2, phenylalanine in step 4 and O-trityl succinicacid hydroxamide in step 6.

Mass found: 606.054.

IC₅₀ (microM): 0.3 (enzyme from E. coli)
0.3 (enzyme from S. aureus).

### Example 44

### {[2-[1-(3-Hydroxycarbamoyl-propionylamino)-2-naphthalen-1-yl-ethyl]-1-(2-thiophen-2-yl-ethyl)-1H-benzoimidazole-5-carbonyl]-amino}-acetic acid methyl ester

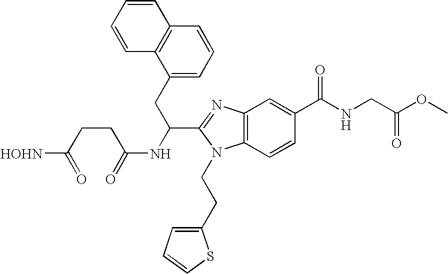

The title compound was prepared according to Method A using thiophenetyl amine in step 2, 1-naphthyl alanine in step 4 and O-trityl succinicacid hydroxamide in step 6.

Mass found: 628.068.

IC₅₀ (microM): 3.1 (enzyme from E. coli)
0.3 (enzyme from S. aureus).

### Example 45

### ({1-Butyl-2-[1-(3-hydroxycarbamoyl-propionylamino)-2-naphthalen-1-yl-ethyl]-1H-benzoimidazole-5-carbonyl}-amino)-acetic acid methyl ester

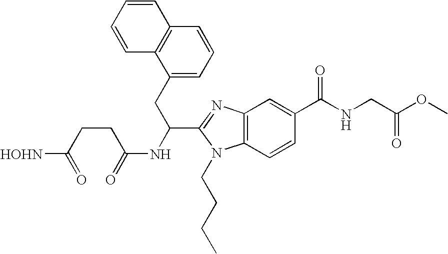

The title compound was prepared according to Method A using butyl amine in step 2, 1-naphthyl alanine in step 4 and O-trityl succinicacid hydroxamide in step 6.

Mass found: 574.110.

IC₅₀ (microM): 5.8 (enzyme from E. coli)
0.3 (enzyme from S. aureus).

### Example 46

### ({1-Benzyl-2-[1-(3-hydroxycarbamoyl-propionylamino)-2-naphthalen-1-yl-ethyl]-1H-benzoimidazole-5-carbonyl}-amino)-acetic acid methyl ester

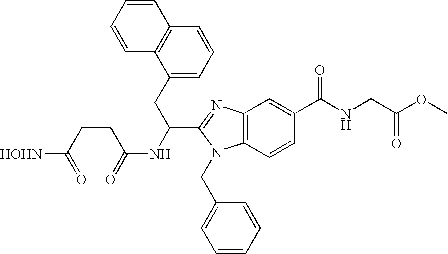

The title compound was prepared according to Method A using benzyl amine in step 2, 1-naphthyl alanine in step 4 and O-trityl succinicacid hydroxamide in step 6.

Mass found: 608.098.

IC₅₀ (microM): 6.5 (enzyme from E. coli)
0.9 (enzyme from S. aureus).

### Example 47

### ({1-Butyl-2-[1-(3-hydroxycarbamoyl-propionylamino)-2-phenyl-ethyl]-1H-benzoimidazole-5-carbonyl}-amino)-acetic acid methyl ester

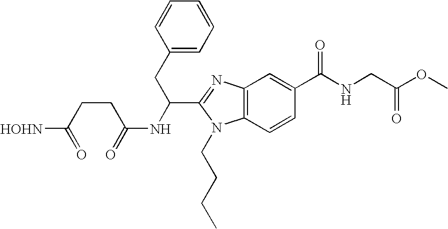

The title compound was prepared according to Method A using benzyl amine in step 2, 1-naphthyl alanine in step 4 and O-trityl succinicacid hydroxamide in step 6.

Mass found: 524.108.

IC₅₀ (microM): 5.3 (enzyme from E. coli)
0.019 (enzyme from S. aureus).

### Example 48

### ({2-[5-Amino-1-(3-mercapto-propionylamino)-pentyl]-1-cyclopropyl-1H-benzoimidazole-5-carbonyl}-amino)-acetic acid methyl ester

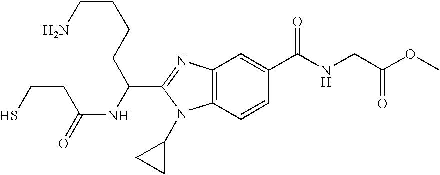

The title compound was prepared according to Method A using cyclopropyl amine in step 2, Boc-lysine in step 4 and S-trityl propionic acid in step 6.

Mass found: 462.087.

IC₅₀ (microM): 3.6 (enzyme from E. coli)
2.0 (enzyme from S. aureus).

### Example 49

### ({2-[5-Amino-1-(3-mercapto-propionylamino)-pentyl]-1-benzyl-1H-benzoimidazole-5-carbonyl}-amino)-acetic acid methyl ester

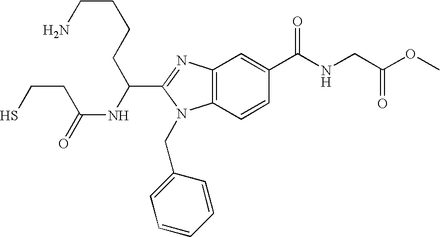

The title compound was prepared according to Method A using benzyl amine in step 2, Boc-lysine in step 4 and S-trityl propionic acid in step 6.

Mass found: 512.084.

IC₅₀ (microM): 5.2 (enzyme from E. coli)
2.4 (enzyme from S. aureus).

### Example 50

### ({1-(2-Amino-ethyl)-2-[1-(3-mercapto-propionylamino)-2-phenyl-ethyl]-1H-benzoimidazole-5-carbonyl}-amino)-acetic acid methyl ester

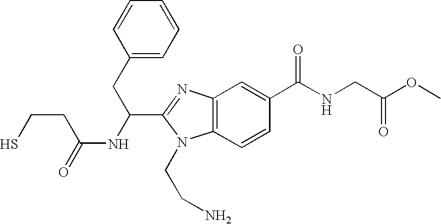

The title compound was prepared according to Method A using ethylenediamine in step 2, phenylalanine in step 4 and S-trityl propionic acid in step 6.

Mass found: 484.068.

IC₅₀ (microM): 7.2 (enzyme from E. coli)
3.8 (enzyme from S. aureus).

### Example 51

### N-{5-Amino-1-[1-benzyl-5-(methoxycarbonylmethyl-carbamoyl)-1H-benzoimidazol-2-yl]-pentyl}-succinamic acid

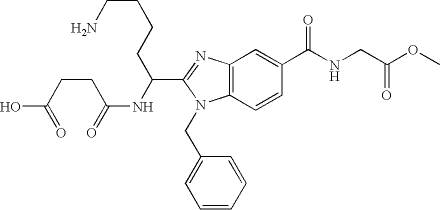

The title compound was prepared according to Method A using benzyl amine in step 2, Boc-lysine in step 4 and succinic acid anhydride in step 6.

Mass found: 524.108.

IC₅₀ (microM): 0.3 (enzyme from E. coli)
0.3 (enzyme from S. aureus).

### Example 52

### N-{1-[1-(2-Amino-ethyl)-5-(methoxycarbonylmethyl-carbamoyl)-1H-benzoimidazol-2-yl]-2-phenyl-ethyl}-succinamic acid

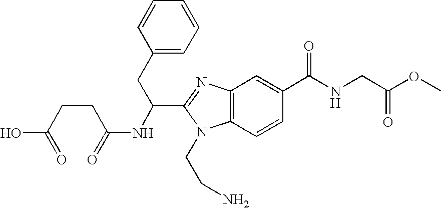

The title compound was prepared according to Method A using ethylenediamine in step 2, phenylalanine in step 4 and succinic acid anhydride in step 6.

Mass found: 496.085.

IC₅₀ (microM): 0.3 (enzyme from E. coli)
0.3 (enzyme from S. aureus).

### Example 53

### ({2-[5-Amino-1-(3-hydroxycarbamoyl-propionylamino)-pentyl]-1-cyclopropyl-1H-benzoimidazole-5-carbonyl}-amino)-acetic acid methyl ester

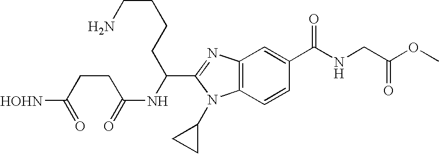

The title compound was prepared according to Method A using cyclopropyl amine in step 2, Boc-lysine in step 4 and O-trityl succinicacid hydroxamide in step 6.

Mass found: 489.159.

IC₅₀ (microM): 0.3 (enzyme from E. coli)
0.3 (enzyme from S. aureus).

### Example 54

### ({2-[5-Amino-1-(3-hydroxycarbamoyl-propionylamino)-pentyl]-1-cyclohexylmethyl-1H-benzoimidazole-5-carbonyl}-amino)-acetic acid methyl ester

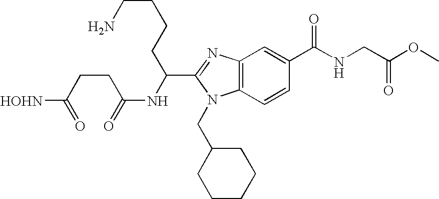

The title compound was prepared according to Method A using cyclohexylmethyl amine in step 2, Boc-lysine in step 4 and O-trityl succinicacid hydroxamide in step 6.

Mass found: 545.136.

IC₅₀ (microM): 0.3 (enzyme from E. coli)
0.3 (enzyme from S. aureus).

### Example 55

### ({2-[5-Amino-1-(3-hydroxycarbamoyl-propionylamino)-pentyl]-1-benzyl-1H-benzoimidazole-5-carbonyl}-amino)-acetic acid methyl ester

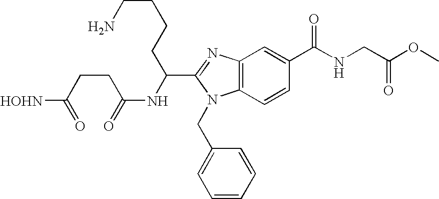

The title compound was prepared according to Method A using benzyl amine in step 2, Boc-lysine in step 4 and O-trityl succinicacid hydroxamide in step 6.

Mass found: 539.080.

IC₅₀ (microM): 0.3 (enzyme from E. coli)
0.3 (enzyme from S. aureus).

### Example 56

### ({1-(2-Amino-ethyl)-2-[1-(3-hydroxycarbamoyl-propionylamino)-2-phenyl-ethyl]-1H-benzoimidazole-5-carbonyl}-amino)-acetic acid methyl ester

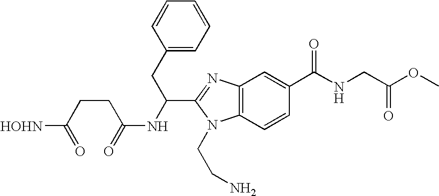

The title compound was prepared according to Method A using ethylenediamine in step 2, phenylalanine in step 4 and O-trityl succinicacid hydroxamide in step 6.

Mass found: 511.084.

IC₅₀ (microM): 2.0 (enzyme from E. coli)
1.6 (enzyme from S. aureus).

### Example 57

### ({1-Cyclopropyl-2-[1-(3-hydroxycarbamoyl-propionylamino)-2-(4-hydroxy-phenyl)-ethyl]-1H-benzoimidazole-5-carbonyl}-amino)-acetic acid methyl ester

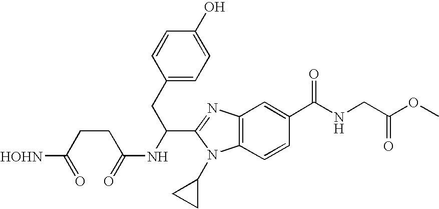

The title compound was prepared according to Method A using cyclopropyl amine in step 2, tyrosine in step 4 and O-trityl succinicacid hydroxamide in step 6.

Mass found: 524.064.

IC₅₀ (microM): 0.3 (enzyme from E. coli)
0.3 (enzyme from S. aureus).

### Example 58

### ({1-Cyclohexylmethyl-2-[1-(3-hydroxycarbamoyl-propionylamino)-2-(4-hydroxy-phenyl)-ethyl]-1H-benzoimidazole-5-carbonyl}-amino)-acetic acid methyl ester

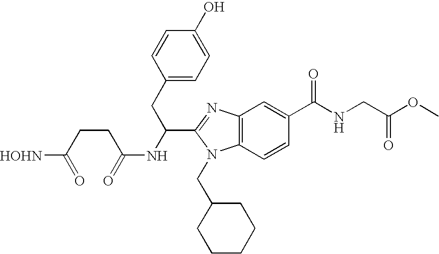

The title compound was prepared according to Method A using cyclohexylmethyl amine in step 2, tyrosine in step 4 and O-trityl succinicacid hydroxamide in step 6.

Mass found: 580.101.

IC₅₀ (microM): 0.3 (enzyme from E. coli)
0.3 (enzyme from S. aureus).

### Example 59

### {[2-[1-(3-Hydroxycarbamoyl-propionylamino)-2-phenyl-ethyl]-1-(2-hydroxy-ethyl)-1H-benzoimidazole-5-carbonyl]-amino}-acetic acid methyl ester

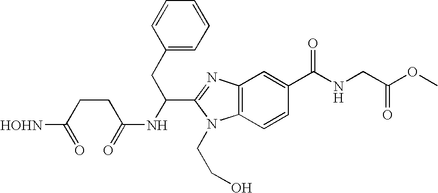

The title compound was prepared according to Method A using aminoethanol in step 2, phenylalanine in step 4 and O-trityl succinicacid hydroxamide in step 6.

Mass found: 512.059.

IC₅₀ (microM): 0.3 (enzyme from E. coli)
0.3 (enzyme from S. aureus).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. Various references are cited herein, the disclosure of which are incorporated by reference in their entireties.

### LITERATURE LIST

Adams, J. M.; Capecchi, M. R. Proc. Natl. Acad. Sci., USA, 1966, 55, 147-155.
Adams, J. M. J. Mol. Biol. 1968, 33, 571-589.
Clements, J. M.; Ayscough, A. P.; Keavey, K.; East, S. P. Curr. Med. Chem. Anti-Infective Agents 2002, 1, 239-249.

Gennaro, A. R.; Gennaro A. L. Remington, The Science and Practice of Pharmacy, 19th ed., Mack Publishing Co., Easton, Pa., 1995.

Giglione, C.; Pierre, M.; Meinnel, T. Mol. Microbiol. 2000, 36, 1197-1205.

Giglione, C.; Meinnel, T. Emerg. Ther. Targets 2001, 5, 41-57.

Green, T. W.; Wuts, P. G. M. Protective Groups In Organic Synthesis, John Wiley & Sons, New York, 1999.

Pei, D. Emerg. Ther. Targets 2001, 5, 23-40.

Renil, M.; Meldal, M. Tetrahedron Lett. 1996, 37. 6185-6188

Yuan, Z.; Trias, J.; White, R. J. Drug Discov. Today 2001, 6, 954-961.

J. Pharm. Sci. 1977, 66, 2.

1. A compound of formula (I) 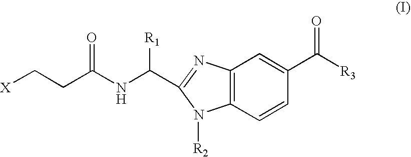
or a pharmaceutical acceptable salt or ester thereof,
wherein
X is —CONHOH, —COOH, —OH, or —SH;
R₁ is selected from the group consisting of C₁₋₆ alkyl, C₃₋₁₀ cycloalkyl, C₁₋₆ alkylmercapto, C₁₋₆ alkylthio-C₁₋₆ alkyl, C₁₋₆ alkylhydroxy, C₁₋₆ alkylcarboxy, C₁₋₆ alkylamide, C₁₋₆ alkylamino, alkylamino-C₁₋₆ alkyl, dialkylamino-C₁₋₆ alkyl, C₁₋₆ alkylamidine, C₁₋₆ alkylguanidine; an unsubstituted or substituted aryl group, an unsubstituted or substituted heteroaryl group, an unsubstituted or substituted C₁₋₆ alkylaryl group, an unsubstituted or substituted C₁₋₆ alkylheteroaryl group and a side chain of a natural alpha amino acid;
with the proviso that R₁ cannot be hydrogen or tert-butyl;
R₂ is selected from the group consisting of C₁₋₆ alkyl, C₂₋₆ alkenyl, C₃₋₁₀ cycloalkyl, C₁₋₆ alkyl-C₃₋₁₀ cycloalkyl, C₃₋₇ heterocycloalkyl, C₁₋₆ alkoxy, C₁₋₆ alkylamino, C₁₋₆ alkylmercapto, C₁₋₆ alkylhydroxy, thio C₁₋₆ alkyl, alkylamino-C₁₋₆ alkyl, dialkylamino-C₁₋₆ alkyl, an unsubstituted or substituted aryl group, an unsubstituted or substituted heteroaryl group, an unsubstituted or substituted C₁₋₆ alkylaryl group and an unsubstituted or substituted C₁₋₆ alkylheteroaryl group;
R₃ is —NHCH (R₄) COR₅, —NR₆R₇, —NHR₇ or —OR₇;
R₄ is selected from the group consisting of hydrogen and a side chain of a natural alpha amino acid;
R₅ is amino, hydroxy, C₁₋₆ alkoxy or —NH—C₁₋₆ alkyl;
R₆ and R₇ are identical or different and are independently of each other selected from the group consisting of C₃₋₇ heterocycloalkyl, an unsubstituted or substituted C₁₋₆ alkyl-C₃₋₇ heterocycloalkyl group, an unsubstituted or substituted aryl group, an unsubstituted or substituted heteroaryl group, an unsubstituted or substituted C₁₋₆ alkylaryl group and an unsubstituted or substituted C₁₋₆ alkylheteroaryl group;
wherein a substituted group is substituted with one, two or three substituents independently selected from halogen, hydroxy, amino, mercapto, nitro, cyano, trifluoromethyl, C₁₋₆ alkyl, C₁₋₆ alkoxy, thioC₁₋₆ alkyl, C₁₋₆ alkylhydroxy, C₁₋₆ alkylamino, alkylamino-C₁₋₆ alkyl and dialkylamino-C₁₋₆ alkyl.

2. A compound according to claim 1, wherein X is —CONHOH.

3. A compound according to claim 1, wherein X is —COOH.

4. A compound according to claim 1, wherein X is selected among —OH and —SH.

5. A compound according to claim 1, wherein R₁ is a side chain of a natural alpha amino acid selected from the group consisting of alanine, arginine, asparagine, aspartic acid, cysteine, glutamin, glutamic acid, glycine, histidine, isoleucine, leucine, lysine, methionine, phenylalanine, serine, threonine, tryptophan, tyrosine and valine.

6. A compound according to claim 1, wherein R₁ is C₁₋₆ alkyl, C₃₋₁₀ cycloalkyl, C₁₋₆ alkylmercapto, C₁₋₆ alkylthio-C₁₋₆ alkyl, C₁₋₆ alkylhydroxy, C₁₋₆ alkylcarboxy, C₁₋₆ alkylamide, C₁₋₆ alkylamino, alkylamino-C₁₋₆ alkyl, dialkylamino-C₁₋₆ alkyl, C₁₋₆ alkylamidine, C₁₋₆ alkylguanidine, an unsubstituted or substituted aryl group, an unsubstituted or substituted heteroaryl group, an unsubstituted or substituted C-6 alkylaryl group or an unsubstituted or substituted C₁₋₆ alkylheteroaryl group.

7. A compound according to claim 1, wherein R₁ is ethyl, isobutyl, 2-(methylsulfanyl)ethyl, 4-aminobutyl, benzyl, 4-hydroxybenzyl, 2-phenylethyl and naphth-1-yl-methyl.

8. A compound according to claim 1, wherein R₂ is selected from the group consisting of C₁₋₆ alkyl, C₃₋₁₀ cycloalkyl, C₁₋₆ alkyl-C₃₋₁₀ cycloalkyl, C₁₋₆ alkylamino, C₁₋₆ alkylhydroxy, an unsubstituted or substituted C₁₋₆ alkylaryl group and an unsubstituted or substituted C₁₋₆ alkylheteroaryl group, wherein a substituted group is substituted with one, two or three substituents independently selected from halogen, hydroxy, amino, mercapto, nitro, cyano, trifluoromethyl, C₁₋₆ alkyl, C₁₋₆ alkoxy, and thioC₁₋₆ alkyl.

9. A compound according to claim 1, wherein R₂ is selected from the group consisting of ethyl, propyl, butyl, cyclopropyl, cyclobutyl, cyclopentyl, cyclohexyl, cyclopropylmethyl, cyclobutylmethyl, cyclohexylmethyl, cyclohexylethyl, aminoethyl, aminopropyl, aminobutyl, hydroxymethyl, hydroxyethyl, hydroxypropyl, hydroxybutyl, an phenyl, fluorosubstituted phenyl, chlorosubstituted phenyl, benzyl, fluorosubstituted benzyl, chlorosubstituted benzyl, thiophenylethyl and furanylmethyl.

10. A compound according to claim 9, wherein R₂ is butyl, cyclopropyl, cyclohexylmethyl, 2-aminoethyl, 2-hydroxyethyl, benzyl, 2-chlorobenzyl, 4-chlorobenzyl, 2,6-difluorobenzyl, 2-thiophen-2-ylethyl or furan-2-ylmethyl.

11. A compound according to claim 1, wherein R₃ is —NHCH(R₄)COR₅.

12. A compound according to claim 1, wherein R₃ is —NHR₇ or —NR₆R₇.

13. A compound according to claim 1, wherein R₃ is —OR₇.

14. A compound according to claim 1, wherein R₄ is a side chain of a natural alpha amino acid selected from the group consisting of alanine, arginine, asparagine, aspartic acid, cysteine, glutamin, glutamic acid, glycine, histidine, isoleucine, leucine, lysine, methionine, phenylalanine, serine, threonine, tryptophan, tyrosine and valine.

15. A compound according to claim 1, wherein R₄ is hydrogen.

16. A compound according to claim 1, wherein R₅ is C₁₋₆ alkoxy.

17. A compound according to claim 1, wherein R₅ is methoxy, ethoxy, propoxy or butoxy.

18. A compound according to claim 1, wherein R₆ or R₇ is C₃₋₇ heterocycloalkyl or an unsubstituted or substituted C₁₋₆ alkyl-C₃₋₇ heterocycloalkyl group.

19. A compound according to claim 1, wherein R₆ or R₇ is an unsubstituted or substituted aryl group, an unsubstituted or substituted heteroaryl group, an unsubstituted or substituted C₁₋₆ alkylaryl group or an unsubstituted or substituted C₁₋₆ alkylheteroaryl group.

20. A compound according to claim 1 selected from the group consisting of
({1-cyclopropyl-2-[1-(3-mercapto-propionylamino)-propyl]-1 H-benzoimidazole-5-carbonyl}-amino)-acetic acid methyl ester,
({1-(4-chloro-benzyl)-2-[1-(3-mercapto-propionylamino)-2-phenyl-ethyl]-1H-benzoimidazole-5-carbonyl}-amino)-acetic acid methyl ester,
N-{1-[1-benzyl-5-(methoxycarbonylmethyl-carbamoyl)-1 H-benzoimidazol-2-yl]-3-methylsulfanyl-propyl}-succinamic acid,
N-{1-[1-butyl-5-(methoxycarbonylmethyl-carbamoyl)-1 H-benzoimidazol-2-yl]-2-phenyl-ethyl}-succinamic acid,
N-{1-[1-furan-2-ylmethyl-5-(methoxycarbonylmethyl-carbamoyl)-1 H-benzoimidazol-2-yl]-2-phenyl-ethyl}-succinamic acid,
N-{1-[1-(4-chloro-benzyl)-5-(methoxycarbonylmethyl-carbamoyl)-1 H-benzoimidazol-2-yl]-2-phenyl-ethyl}-succinamic acid,
N-{1-[1-cyclopropyl-5-(methoxycarbonylmethyl-carbamoyl)-1 H-benzoimidazol-2-yl]-3-phenyl-propyl}-succinamic acid,
N-{1-[1-cyclohexylmethyl-5-(methoxycarbonylmethyl-carbamoyl)-1 H-benzoimidazol-2-yl]-3-phenyl-propyl}-succinamic acid,
N-{1-[1-(2-chloro-benzyl)-5-(methoxycarbonylmethyl-carbamoyl)-1 H-benzoimidazol-2-yl]-3-phenyl-propyl}-succinamic acid,
N-{1-[1-cyclopropyl-5-(methoxycarbonylmethyl-carbamoyl)-1 H-benzoimidazol-2-yl]-propyl}-succinamic acid,
N-{1-[1-furan-2-ylmethyl-5-(methoxycarbonylmethyl-carbamoyl)-1H-benzoimidazol-2-yl]-propyl}-succinamic acid,
N-{1-[1-benzyl-5-(methoxycarbonylmethyl-carbamoyl)-1 H-benzoimidazol-2-yl]-propyl}-succinamic acid,
N-{1-[1-cyclopropyl-5-(methoxycarbonylmethyl-carbamoyl)-1H-benzoimidazol-2-yl]-3-methyl-butyl}-succinamic acid,
N-{1-[1-butyl-5-(methoxycarbonylmethyl-carbamoyl)-1 H-benzoimidazol-2-yl]-3-methyl-butyl}-succinamic acid,
N-{1-[1-benzyl-5-(methoxycarbonylmethyl-carbamoyl)-1 H-benzoimidazol-2-yl]-3-methyl-butyl}-succinamic acid,
N-{1-[1-cyclopropyl-5-(methoxycarbonylmethyl-carbamoyl)-1 H-benzoimidazol-2-yl]-3-methylsulfanyl-propyl}-succinamic acid,
N-{1-[5-(methoxycarbonylmethyl-carbamoyl)-1-(2-thiophen-2-yl-ethyl)-1 H-benzoimidazol-2-yl]-2-naphthalen-1-yl-ethyl}-succinamic acid,
N-{1-[1-butyl-5-(methoxycarbonylmethyl-carbamoyl)-1 H-benzoimid zol-2-yl]-2-naphthalen-1-yl-ethyl}-succinamic acid,
({2-[5-amino-1-(3-mercapto-propionylamino)-pentyl]-1-cyclohexylmethyl-1 H-benzoimidazole-5-carbonyl}-amino)-acetic acid methyl ester,
({1-cyclopropyl-2-[2-(4-hydroxy-phenyl)-1-(3-mercapto-propionylamino)-ethyl]-1H-benzoimidazole-5-carbonyl}-amino)-acetic acid methyl ester,
({1-cyclohexylmethyl-2-[2-(4-hydroxy-phenyl)-1-(3-mercapto-propionylamino)-ethyl]-1 H-benzoimidazole-5-carbonyl}-amino)-acetic acid methyl ester,
({1-(2-hydroxy-ethyl)-2-[1-(3-mercapto-propionylamino)-2-phenyl-ethyl]-1H-benzoimidazole-5-carbonyl}-amino)-acetic acid methyl ester,
N-{5-amino-1-[1-cyclopropyl-5-(methoxycarbonylmethyl-carbamoyl)-1 H-benzoimidazol-2-yl]-pentyl}-succinamic acid,
N-{5-amino-1-[1-cyclohexylmethyl-5-(methoxycarbonylmethyl-carbamoyl)-1 H-benzoimidazol-2-yl]-pentyl}-succinamic acid,
N-[1-[1-cyclopropyl-5-(methoxycarbonylmethyl-carbamoyl)-1 H-benzoimidazol-2-yl]-2-(4-hydroxyphenyl)-ethyl]-succinamic acid,
N-[1-[1-cyclohexylmethyl-5-(methoxycarbonylmethyl-carbamoyl)-1 H-benzoimidazol-2-yl]-2-(4-hydroxyphenyl)-ethyl]-succinamic acid,
N-{1-[1-(2-hydroxy-ethyl)-5-(methoxycarbonylmethyl-carbamoyl)-1 H-benzoimidazol-2-yl]-2-phenyl-ethyl}-succinamic acid,
N-{1-[1-(2-hydroxy-ethyl )-5-(methoxycarbonylmethyl-carbamoyl)-1 H-benzoimidazol-2-yl]-3-methylbutyl}-succinamic acid,
({1-cyclopropyl-2-[1-(3-hydroxycarbamoyl-propionylamino)-propyl]-1 H-benzoimidazole-5-carbonyl}-amino)-acetic acid methyl ester,
N-{1-[1-benzyl-5-(methoxycarbonylmethyl-carbamoyl)-1 H-benzoimidazol-2-yl]-2-naphthalen-1-yl-ethyl}-succinamic acid,
({1-furan-2-ylmethyl-2-[1-(3-hydroxycarbamoyl-propionylamino)-propyl]-1-H-benzoimidazole-5-carbonyl}-amino)-acetic acid methyl ester,
({1-benzyl-2-[1-(3-hydroxycarbamoyl-propionylamino)-propyl]-1 H-benzoimidazole-5-carbonyl}-amino)-acetic acid methyl ester,
({1-cyclopropyl-2-[1-(3-hydroxycarbamoyl-propionylamino)-3-methyl-butyl]-1 H-benzoimidazole-5-carbonyl}-amino)-acetic acid methyl ester,
({1-butyl-2-[1-(3-hydroxycarbamoyl-propionylamino)-3-methyl-butyl]-1 H-benzoimidazole-5-carbonyl}-amino)-acetic acid methyl ester,
({1-benzyl-2-[1-(3-hydroxycarbamoyl-propionylamino)-3-methyl-butyl]-1 H-benzoimidazole-5-carbonyl}-amino)-acetic acid methyl ester,
({1-cyclopropyl-2-[1-(3-hydroxycarbamoyl-propionylamino)-3-methylsulfanyl-propyl]-1 H-benzoimidazole-5-carbonyl}-amino)-acetic acid methyl ester,
({1-cyclohexylmethyl-2-[1-(3-hydroxycarbamoyl-propionylamino)-3-methylsulfanyl-propyl]-1 H-benzoimidazole-5-carbonyl}-amino)-acetic acid methyl ester,
({1-benzyl-2-[1-(3-hydroxycarbamoyl-propionylamino)-3-methylsulfanyl-propyl]-1 H-benzoimidazole-5-carbonyl}-amino)-acetic acid methyl ester,
({1-furan-2-ylmethyl-2-[1-(3-hydroxycarbamoyl-propionylamino)-2-phenyl-ethyl]-1 H-benzoimidazole-5-carbonyl}-amino)-acetic acid methyl ester,
({1-(4-chloro-benzyl)-2-[1-(3-hydroxycarbamoyl-propionylamino)-2-phenyl-ethyl]-1 H-benzoimidazole-5-carbonyl}-amino)-acetic acid methyl ester,
({1-cyclopropyl-2-[1-(3-hydroxycarbamoyl-propionylamino)-3-phenyl-propyl]-1 H-benzoimidazole-5-carbonyl}-amino)-acetic acid methyl ester,
({1-cyclohexylmethyl-2-[1-(3-hydroxycarbamoyl-propionylamino)-3-phenyl-propyl]-1 H-benzoimidazole-5-carbonyl}-amino)-acetic acid methyl ester,
({1-(2-chloro-benzyl)-2-[1-(3-hydroxycarbamoyl-propionylamino)-2-phenyl-ethyl]-1 H-benzoimidazole-5-carbonyl}-amino)-acetic acid methyl ester,
{[2-[1-(3-hydroxycarbamoyl-propionylamino)-2-naphthalen-1-yl-ethyl]-1-(2-thiophen-2-yl-ethyl)-1 H-benzoimidazole-5-carbonyl]-amino}-acetic acid methyl ester,
({1-butyl-2-[1-(3-hydroxycarbamoyl-propionylamino)-2-naphthalen-1-yl-ethyl]-1 H-benzoimidazole-5-carbonyl}-amino)-acetic acid methyl ester,
({1-benzyl-2-[1-(3-hydroxycarbamoyl-propionylamino)-2-naphthalen-1-yl-ethyl]-1 H-benzoimidazole-5-carbonyl}-amino)-acetic acid methyl ester,
({1-butyl-2-[1-(3-hydroxycarbamoyl-propionylamino)-2-phenyl-ethyl]-1 H-benzoimidazole-5-carbonyl}-amino)-acetic acid methyl ester,
({2-[5-amino-1-(3-mercapto-propionylamino)-pentyl]-1-cyclopropyl-1H-benzoimidazole-5-carbonyl}-amino)-acetic acid methyl ester,
({2-[5-amino-1-(3-mercapto-propionylamino)-pentyl]-1-benzyl-1H-benzoimidazole-5-carbonyl}-amino)-acetic acid methyl ester,
({1-(2-amino-ethyl)-2-[1-(3-mercapto-propionylamino)-2-phenyl-ethyl]-1 H-benzoimidazole-5-carbonyl}-amino)-acetic acid methyl ester,
N-{5-amino-1-[1-benzyl-5-(methoxycarbonylmethyl-carbamoyl)-1 H-benzoimidazol-2-yl]-pentyl}-succinamic acid,
N-{1-[1-(2-amino-ethyl)-5-(methoxycarbonylmethyl-carbamoyl)-1 H-benzoimidazol-2-yl]-2-phenyl-ethyl}-succinamic acid,
({2-[5-amino-1-(3-hydroxycarbamoyl-propionylamino)-pentyl]-1-cyclopropyl-1 H-benzoimidazole-5-carbonyl}-amino)-acetic acid methyl ester,
({2-[5-amino-1-(3-hydroxycarbamoyl-propionylamino)-pentyl]-1-cyclohexylmethyl-1 H-benzoimidazole-5-carbonyl}-amino)-acetic acid methyl ester,
({2-[5-amino-1-(3-hydroxycarbamoyl-propionylamino)-pentyl]-1-benzyl-1 H-benzoimidazole-5-carbonyl}-amino)-acetic acid methyl ester,
({1-(2-amino-ethyl)-2-[1-(3-hydroxycarbamoyl-propionylamino)-2-phenyl-ethyl]-1 H-benzoimidazole-5-carbonyl}-amino)-acetic acid methyl ester,
({1-cyclopropyl-2-[1-(3-hydroxycarbamoyl-propionylamino)-2-(4-hydroxy-phenyl)-ethyl]-1 H-benzoimidazole-5-carbonyl}-amino)-acetic acid methyl ester,
({1-cyclohexylmethyl-2-[1-(3-hydroxycarbamoyl-propionylamino)-2-(4-hydroxy-phenyl)-ethyl]-1 H-benzoimidazole-5-carbonyl}-amino)-acetic acid methyl ester,
{[2-[1-(3-hydroxycarbamoyl-propionylamino)-2-phenyl-ethyl]-I-(2-hydroxy-ethyl)-I H-benzoimidazole-5-carbonyl]-amino}-acetic acid methyl ester; and stereoisomers thereof.

21. A compound according to claim 1, which in a PDF assay exhibits an IC₅₀ value of less than 500 μM.

22-25. (canceled)

26. A pharmaceutical composition comprising, as an active ingredient, a compound according to claim 1 or a pharmaceutically acceptable salt thereof together with a pharmaceutically acceptable carrier or diluent.

27. A pharmaceutical composition according to claim 26 comprising a second active substance having antibacterial activity.

28. A pharmaceutical composition according to claim 26, wherein the composition is in unit dosage form comprising from about 1 μg to about 1000 mg of the active substance or a pharmaceutically acceptable salt or ester thereof.

29-30. (canceled)

31. A pharmaceutical composition claim according to claim 1 for oral, nasal, transdermal, pulmonal or parenteral administration.

32. A method for the treatment of an ailment, the method comprising administering to a subject in need thereof an effective amount of a compound according to claim 1 or a pharmaceutically acceptable salt thereof.

33. A method according to claim 32, wherein the effective amount of a compound or a pharmaceutical acceptable salt or ester thereof is in the range of from about 1 μg to about 1000 μg per day.

34-38. (canceled)

39. A method for the treatment of a patient suffering from or susceptible to a bacterial infection, the method comprising administering to the patient an effective amount of a compound of claim 1.

40. The method of claim 39 wherein the patient is suffering from a bacterial infection.

41. The method of claim 39 wherein the patient has been identified and selected for treatment as suffering from a bacterial infection and the compound is administered to the selected patient.

42. The method of claim 39 wherein the patient is suffering from an infection an organism belonging any of the genera Staphylococcus, Enterococcus, Streptococcus, Haemophilus, Moraxella, Escherichia, Mycobacterium, Mycoplasma, Pseudomonas, Chlamydia, Rickettsia, Klebsiella, Shigella, Salmonella, Bordetella, Clostridium, Helicobacter, Campylobacter, Legionella and Neisseria.

43. The method of claim 39 wherein the patient is suffering from an infection associated with an organism belonging to the group consisting of Staphylococcus aureus, Staphylococcus epidermidis, Enterococcus faecium, Enterococcus faecalis, Streptococcus pneumoniae, Haemophilus influenzae, Moraxella catarrhalis, Escherichia coli, Mycobacterium tuberculosis, Mycobacterium ranae, Mycoplasma pneumoniae, Pseudomonas aeruginosa, Chlamydia, Rickettsiae, Klebsiella pneumoniae, Shigella flexneri, Salmonella typhimurium, Bordetella pertussis, Clostridia perfringens, Helicobacterpylori, Campylobacter jejuni, Legionella pneumophila and Neisseria gonorrhoeae.
